(19) United States
(12) Patent Application Publication
(10) Pub. No.: US 2024/0366672 A1
(43) Pub. Date: Nov. 7, 2024

(54) CHIMERIC ANTIGEN RECEPTOR AND USE THEREOF

(71) Applicant: ORICELL THERAPEUTICS CO., LTD., Shanghai (CN)

(72) Inventors: Hao GUO, Shanghai (CN); Siye CHEN, Shanghai (CN); Xiaowen HE, Shanghai (CN); Huijiao LI, Shanghai (CN); Youguo LING, Shanghai (CN); Yue YANG, Shanghai (CN); Yanhong XU, Shanghai (CN); Qi YANG, Shanghai (CN); Zhifeng XU, Shanghai (CN); Xiaopei LI, Shanghai (CN); Huanfeng YANG, Shanghai (CN)

(73) Assignee: ORICELL THERAPEUTICS CO., LTD., Shanghai (CN)

(21) Appl. No.: 18/686,716

(22) PCT Filed: Aug. 27, 2021
(86) PCT No.: PCT/CN2021/115057

§ 371 Date: Feb. 26, 2024

(51) Int. Cl. A61K 35/17; A61K 39/00; A61P 35/00; C07K 14/725; C07K 16/30; C12N 15/86

(52) U.S. Cl. CPC A61K 35/17; A61K 39/4611; A61K 39/4631; A61K 39/464474; A61P 35/00; C07K 14/7051; C07K 16/303; C12N 15/86; A61K 2239/13; A61K 2239/21; A61K 2239/31; A61K 2239/53; C07K 2317/565; C07K 2317/622; C07K 2319/02; C07K 2319/03; C12N 2740/15043

(57) ABSTRACT

The present application relates to a chimeric antigen receptor (CAR), comprising a GPC3 binding domain, a transmembrane domain, a costimulatory domain and an intracellular signaling domain, the GPC3 binding domain comprises an antibody or a fragment thereof which specifically binding to GPC3, the antibody comprises a light chain complementary determining region 1 (LCDR1), a light chain complementary determining region 2 (LCDR2) and a light chain complementary determining region 3 (LCDR3), the amino acid sequence of the LCDR1 is as set forth in SEQ ID NO: 16, the amino acid sequence of the LCDR2 is as set forth in SEQ ID NO: 17 and the amino acid sequence of the LCDR3 is as set forth in SEQ ID NO: 18. The present application also relates to an isolated nucleic acid encoding the CAR, a vector comprising the nucleic acid, an immune effector cell comprising the nucleic acid or the vector and a preparation method thereof as well as a use of the CAR.

### TECHNICAL FIELD

The present application relates to the field of biomedicine, and particularly relates to a chimeric antigen receptor and a use thereof.

### BACKGROUND

Tumor is a serious threat to human health, of which hepatic carcinoma is a kind of malignant tumor with extensive harm. Hepatic carcinoma caused by hepatitis B virus has a feature of long latent period. It always has developed to the advanced stage once discovered, and it progresses very rapidly after the onset and the therapeutic prognosis is poor.

Glypican 3 (GPC3) is a kind of heparan sulfate proteoglycan on the surface of cell membrane, which exists in various tumors and especially common in hepatic carcinoma. In recent years, with the progress of molecular biology, genomics and proteomics, a series of molecular targeted drugs have been developed in succession for the treatment of hepatic carcinoma. However, along with the rapid development of immunology, in light of such a discovery that the recognition specificity of cytotoxic lymphocytes (CTL) on target cells depends on T lymphocyte receptors (TCR), it is further investigated to fuse the scFv of antibodies against tumor cell associated antigen with T lymphocyte receptors to form chimeric antigen receptors (CAR), which are genetically modified on the surface of T lymphocytes by means of lentivirus infection for the treatment of tumors. This kind of CAR-T lymphocytes can selectively target T lymphocytes to tumor cells in a nonrestrictive way of major histocompatibility complex (MHC) and specifically kill the tumor cells. However, adoptive immunotherapy based on immune effector cells has achieved some effects in some tumors, but its efficacies in most tumors are not satisfactory.

### SUMMARY OF THE INVENTION

The present application provides a chimeric antigen receptor (CAR), wherein the CAR comprises a GPC3 binding domain, a transmembrane domain, a costimulatory domain and an intracellular signaling domain, the GPC3 binding domain comprises an antibody or a fragment thereof which specifically binding to GPC3, wherein the antibody comprises a light chain complementary determining region 1 (LCDR1), a light chain complementary determining region 2 (LCDR2) and a light chain complementary determining region 3 (LCDR3), the amino acid sequence of the LCDR1 is as set forth in SEQ ID NO: 16, the amino acid sequence of the LCDR2 is as set forth in SEQ ID NO: 17 and the amino acid sequence of the LCDR3 is as set forth in SEQ ID NO: 18.

In some embodiments, the antibody comprises a heavy chain complementary determining region 1 (HCDR1), a heavy chain complementary determining region 2 (HCDR2) and a heavy chain complementary determining region 3 (HCDR3), the amino acid sequence of the HCDR1 is as set forth in SEQ ID NO: 19, the amino acid sequence of the HCDR2 is as set forth in SEQ ID NO: 20 and the amino acid sequence of the HCDR3 is as set forth in SEQ ID NO: 21.

In some embodiments, the antibody comprises a heavy chain variable region, and the amino acid sequence of the heavy chain variable region is as set forth in SEQ ID NO: 29.

In some embodiments, the antibody comprises a light chain variable region, and the amino acid sequence of the light chain variable region is as set forth in SEQ ID NO: 25.

In some embodiments, the antibody is a single-chain antibody.

In some embodiments, the antibody comprises an amino acid sequence as set forth in SEQ ID NO: 30.

In some embodiments, the transmembrane domain comprises a transmembrane domain derived from a protein selected from: CD28, CD3e, CD45, CD4, CD5, CD8, CD9, CD16, CD22, CD33, CD37, CD64, CD80, CD86, CD134, CD137 and CD154.

In some embodiments, the transmembrane domain comprises an amino acid sequence as set forth in SEQ ID NO: 36.

In some embodiments, the costimulatory domain comprises a costimulatory domain of a protein selected from: CD137, CD28, 4-1BB, OX-40 and ICOS.

In some embodiments, the costimulatory domain comprises an amino acid sequence as set forth in SEQ ID NO: 37.

In some embodiments, the intracellular signaling domain comprises a signaling domain derived from CD3ζ.

In some embodiments, the intracellular signaling domain comprises an amino acid sequence as set forth in SEQ ID NO: 38.

In some embodiments, the CAR further comprises a hinge region, which is linked to the GPC3 binding domain and the transmembrane domain.

In some embodiments, the hinge region comprises an amino acid sequence as set forth in SEQ ID NO: 39.

In some embodiments, the CAR further comprises a linking signal peptide.

In some embodiments, the signal peptide comprises an amino acid sequence as set forth in SEQ ID NO: 40.

In some embodiments, a nucleic acid molecule encoding the CAR is also linked to a promoter.

In some embodiments, the promoter is a constitutive promoter.

In some embodiments, the promoter comprises a nucleotide sequence as set forth in SEQ ID NO: 41.

In some embodiments, the CAR comprises an amino acid sequence as set forth in any one of SEQ ID NOs: 31-34.

The present application also provides an isolated nucleic acid molecule, which encodes the CAR of the present application.

In some embodiments, the isolated nucleic acid molecule encoding the CAR comprises a nucleic acid sequence as set forth in any one of SEQ ID NOs: 42-45.

The present application also provides a vector, which comprises the nucleic acid molecule of the present application.

In some embodiments, the vector is selected from a plasmid, a retrovirus vector and a lentiviral vector.

The present application also provides an immune effector cell, which comprises the CAR of the present application, the nucleic acid molecule of the present application, or the vector of the present application.

In some embodiments, the immune effector cell is selected from T lymphocytes.

The present application also provides a method of preparing the immune effector cell of the present application, which comprises introducing the vector of the present application into the immune effector cell of the present application.

The present application also provides a composition, which comprises the immune effector cell of the present application.

The present application also provides a use of the CAR of the present application, the nucleic acid molecule of the present application, the vector of the present application or the immune effector cell of the present application in preparing a medicament, wherein the medicament is used to treat a disease or disorder related to the expression of GPC3.

The present application also provides a method of treating the disease or disorder related to the expression of GPC3, which comprises administering to a patient or a subject the CAR of the present application, the nucleic acid molecule of the present application, the vector of the present application or the immune effector cell of the present application.

The present application also provides the CAR, the nucleic acid molecule, the vector or the immune effector cell, which is used to treat a disease or disorder related to the expression of GPC3.

In some embodiments, the disease or disorder related to the expression of GPC3 is a cancer or a malignant tumor.

Other aspects and advantages of the present application will be readily conceived by those skilled in the art from the following detailed description. Only the exemplary embodiments of the present application are shown and described in the following detailed description. As will be appreciated by those skilled in the art, the disclosure of the present application allows persons skilled in the art to modify the disclosed embodiments without departing from the spirit and scope of the invention involved in the present application. Accordingly, the drawings and the description in the specification of the present application are merely exemplary, and not restrictive.

### BRIEF DESCRIPTION OF THE DRAWINGS

Specific features of the invention involved in this application are set forth in the appended claims. The features and advantages of the invention involved in the present application can be better understood by referring to the exemplary embodiments detailed hereinafter and the accompanying drawings. A brief description of the drawings is as follows:

FIG. 1 shows the linking order of various parts of a chimeric antigen receptor in the CAR plasmid.

FIG. 2 shows the amplification times of T cells infected by 204A-BBz.

FIG. 3 shows the amplification times of T cells infected by L1H2-BBz/L1H6-BBz/L2H6-BBz/GC33-BBz.

FIG. 4 shows the killing effect of 204A-CAR-T on HepG2 cells.

FIG. 5 shows the killing effect of L1H2/L1H6/L2H6-CAR-T on HepG2 cells.

FIG. 6 shows the amplification times of 204A-CAR-T stimulated repeatedly by Huh7 target cells.

FIG. 7 shows the amplification times of L1H2/L1H6/L2H6-CAR-T stimulated repeatedly by Huh7 target cells.

FIG. 8A-8C show the comparison of inhibition of tumors and prevention of recurrence in L1H2-BBz and GC33-BBz mice.

FIG. 9A-9C show the changes in the body weight of L1H2-BBz and GC33-BBz mice.

FIG. 10 shows the changes of cytokines in mice treated with L1H2-BBz and GC33-BBz.

FIG. 11A-11B show the changes of cell level in mice treated with L1H2-BBz and GC33-BBz (A shows CD8+ cells; B shows CD4+ cells).

FIG. 12 shows the survival curve of mice treated with L1H2-BBz and GC33-BBz.

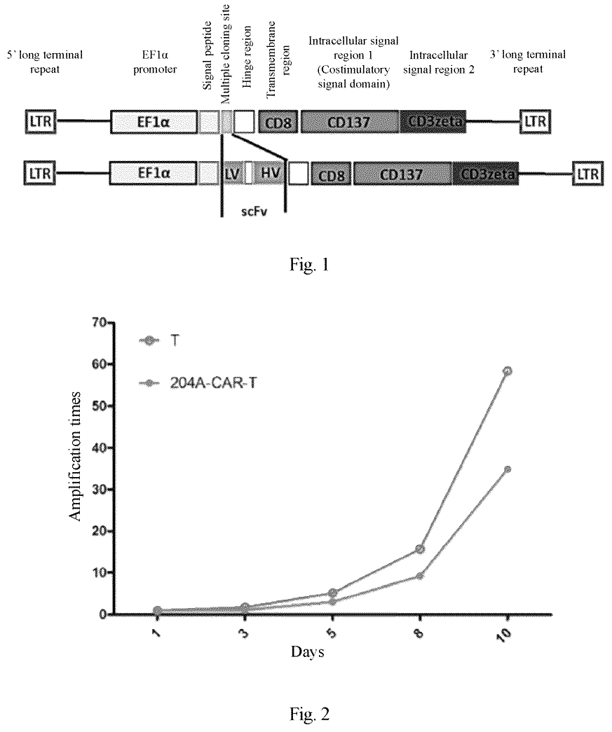

### DETAILED DESCRIPTION

The embodiments of the present invention are described below by way of specific examples, and those skilled in the art can readily appreciate other advantages and effects of the present invention from the disclosure of the present specification.

The present application is further described below: in the present invention, unless otherwise indicated, scientific and technical terms used herein have meanings commonly understood by persons skilled in the art. In addition, the used terms and laboratory operating steps related to protein and nucleic acid chemistry, molecular biology, cell and tissue culture, microbiology, immunology are all terms and conventional steps widely used in corresponding fiels. At the same time, to better understand the present invention, the definitions and explanations of related terms are provided below.

In the present application, the term “chimeric antigen receptor (CAR)” generally refers to the antigen receptor formed from the fusion between the antigen binding region of an antibody recognizing the tumor associated antigen (TAA) and the “immunoreceptor tyrosine-based activation motifs (ITAM, generally CD3ζ or FcεRIγ)” of an intracellular signal domain. The basic structure of CAR includes one tumor associated antigen (TAA) binding region (generally deriving from the scFv of the antigen binding region of a monoclonal antibody), one extracellular hinge region, one transmembrane region and one intracellular immunoreceptor tyrosine-based activation motif (ITAM).

In the present application, the term “GPC3” generally refers to glypican 3 (GPC3), which is a heparan sulfate proteoglycan on the surface of cell membrane and exists in various tumors, and especially common in hepatic carcinoma. GPC3 protein is highly expressed in the liver at fetal period. The abnormal expression of GPC3 protein after birth is closely related to the occurrence and development of tumors. GPC3 protein is highly expressed in primary hepatic carcinoma (PHC), but expressed lowly or moderately in other tumors or benign liver diseases. For example, the GPC3 protein in the present application may include human GPC3 protein.

In the present application, the term “binding domain” generally refers to a given target epitope or a given target site (specifically) binding to a target molecule (antigen), or a domain interacting with the given target epitope or the given target site or recognizing the given target epitope or the given target site.

In the present application, the term “specifically binding” generally refers to a measurable and reproducible interaction, e.g., the binding between a target and an antibody, which can determine the presence of the target in the presence of a heterogeneous group of molecules (including biological molecules). For example, an antibody specifically binding to a target (which may be an epitope) is an antibody which binds to this target with larger affinity, avidity, easier, and/or for a longer period of time than it binds to other targets. In some embodiments, an antibody specifically binds to an epitope on a protein, and the epitope is conservative in different species of protein. In another embodiment, specific binding may include, but not require exclusive binding.

In the present application, the term “CDR” generally refers to an area of an antibody variable domain, also known as a complementary determining region, of which the sequence is highly variable and/or forms a structure-defining ring. In general, an antibody includes six CDRs; three in VH (HCDR1, HCDR2, HCDR3), and three in VL (LCDR1, LCDR2, LCDR3). In native antibodies, HCDR3 and LCDR3 display the most diversity of the six CDRs, and in particular, HCDR3 is considered to play a special role in conferring a fine specificity to the antibody. See, for example, Xu et al, Immunity 13:37-45 (2000); Johnson and Wu, in Methods in Molecular Biology 248:1-25 (Lo, ed., Human Press, Totowa, N.J., 2003). In fact, naturally occurring camel antibodies only composed of heavy chains function normally and are stable in the absence of light chains. See, for example, Hamers-Casterman et al., Nature 363:446-448 (1993); Sheriff et al, Nature Struct. Biol. 3:733-736 (1996).

In the present application, the term “variable region” generally refers to an amino terminal domain of the heavy chain or light chain of an antibody. The variable regions of a heavy chain and a light chain may be called heavy chain variable region (VH) and light chain variable region (VL), respectively. These areas are generally portions with greatest changes in an antibody (relative to the same type of other antibodies), and comprise antigen binding sites.

In the present application, the term “antibody” generally refers to an immunoglobulin or a fragment thereof or a derivative thereof, including any polypeptides containing antigen binding sites, produced either in vitro or in vivo. This term includes, but not limited to, a polyclonal, monoclonal, monospecific, multispecific, nonspecific, humanized, single-chain, chimeric, synthesized, recombinant, hybridized, mutated and transplanted antibody. Unless otherwise being modified by the term “intact”, e.g., in the term “intact antibody”, for the purpose of the present invention, the term “antibody” also includes a fragment of the antibody, e.g., Fab, F (ab′)₂, Fv, scFv, Fd, dAb and other antibody fragments retaining the antigen-binding function (e.g., specifically binding to GPC3). In general, such fragments should include antigen binding domains. An essential 4-chain antibody unit is a hereotetramer glycoprotein composed of two same light chains (L) and two same heavy chains (H). IgM antibody is composed of 5 essential hereotetramer units and another polypeptide called J chain, and contains 10 antigen binding sites; while IgA antibody includes 2-5 essential 4-chain units which can polymerize with the J chain to form a multivalent combination. With regard to IgG, the 4-chain unit is generally about 150,000 Daltons. Each L chain is linked to an H chain through one covalent disulfide bond, and two H chains are linked to each other through one or more disulfide bonds depending on the isotype of H chain. Each of H and L chains also has regularly spaced intrachain disulfide bridge bonds. Each H chain has a variable domain (VH) at its N terminal, followed by three constant domains (CH) respectively for α and γ chains, and followed by four CH domains respectively for μ and ε isotypes. Each L chain has a variable domain (VL) at its N terminal, and has a constant domain at the other terminal. VL corresponds to VH, and CL corresponds to the first constant domain (CH1) of the heavy chain. Specific amino acid residues are considered to form an interface between the light chain and heavy chain variable domains. VH matches with VL to form a single antigen binding site. For the structures and properties of different kinds of antibodies, see for example Basic and Clinical Immunology, 8th Edition, Daniel P. Sties, Abba I. Terr and Tristram G. Parsolw (eds), Appleton & Lange, Norwalk, Conn., 1994, page 71 and Chapter 6. An L chain from any vertebrate species may be classified into one of two distinctly different types based on the amino acid sequence of its constant domain, called as κ and λ. Depending on the amino acid sequence of its heavy chain (CH) constant domain, immunoglobulin can be classified into different types or isotypes. There are five classes of immunoglobulin: IgA, IgD, IgE, IgG and IgM, which have heavy chains being named as α, δ, ε, γ and μ, respectively. Based on relatively small differences in terms of CH sequences and functions, γ and α types are further divided into subtypes. For example, human expresses the following subtypes: IgG1, IgG2A, IgG2B, IgG3, IgG4, IgA1 and IgK1.

In the present application, the term “single-chain antibody” refers to a single-chain polypeptide containing one or more antigen-binding sites. In addition, although the H and L chains of a Fv fragment are encoded by different genes, they can linked together directly or through a peptide. For example, by means of recombination, H and L chains can be linked with a synthetic linker to form a single protein chain (known as a single-chain antibody, sAb; Bird et al. 1988 Science 242:423-426; and Huston et al. 1988 PNAS 85:5879-5883). This single-chain antibody is also included in the term “antibody”, which can be used as the binding determinant in the design and manufacturing of multispecific binding molecules.

In the present application, the term “transmembrane domain” generally refers to a polypeptide or a protein, the polypeptide or protein is encoded by at least one exon comprising an extracellular region, a transmembrane region and an intracellular region on DNA level. The transmembrane domain generally includes three different structural regions: an extracellular region at N terminal, a conservative transmembrane extension region in the middle, and a cytoplasmic region at C terminal. The transmembrane domain may also include an intracellular region or a cytoplasmic region.

In the present application, the term “costimulatory” generally refers to the source from which lymphocytes activate the second signal, which is generally produced from the interaction between the surface costimulatory molecules of immune cells (between T cells/B cells or between antigen-presenting cells/T cells) participating in adaptive immunity and their receptors. For example, the complete activation of T cells depends on the action of double signals and cytokines. The first signal of T cell activation derives from the specific binding of its receptor TCR with the antigeno, that is, the recognization of T cells on the antigen; the second signal of T cell activation derives from the costimulatory molecules, that is, the interaction between the costimulatory molecules on APC and corresponding receptors on the surface of T cells.

In the present application, the term “costimulatory domain” generally refers to any amino acid sequences that can interact with costimulatory molecules to produce costimulation.

In the present application, the term “intracellular signaling domain” generally refers to the intracellular part of a molecule. The intracellular signaling domain produces signals that promote the immune effector function of cells containing CAR, e.g., CART cells. For example, in CART cells, the examples of immune effector function include cell lysis activity and auxiliary activity, including the secretion of cytokines. In some embodiments, the intracellular signal domain transduces the effector function signal and instructs the cells to perform specialized functions. Although the whole intracellular signaling domain can be used, in most cases, it is not necessary to use the whole chain. In terms of using the truncated part of the intracellular signaling domain, such a truncated part can be used in stead of a full chain, as long as it can transduce the effector function signal. Thus, the term intracellular signaling domain is intended to include any truncated part of the intracellular signaling domain that is sufficient to transducer the effector function signal.

In the present application, the term “CD”, i.e., Cluster of differentiation, is generally used to recognize cell surface molecules used as immunological antigen markers. CD molecules have many uses and generally used as important receptors or ligands of cells. Partial CDs can take part in the signal cascade of cells so as to change the behavior of cells, while some CD proteins are irrelevant to cell signaling, but may have other functions, for example, cell adhesion. As of Apr. 21, 2016, the total number of human CD molecules is 371, e.g., CD28, CD3e, CD45, CD4, CD5, CD8, CD9, CD16, CD22, CD33, CD37, CD64, CD80, CD86, CD134, CD137 and CD154 as sources of transmembrane domains, and CD137, CD28, CD134 (OX-40) and CD278 (ICOS) as sources of costimulatory domains in the present application.

In the present application, the term “CD137”, also known as 4-1BB, generally refers to a member of the tumor necrosis factor (TNF) receptor family, which is encoded genetically by the tumor necrosis factor receptor superfamily member 9 (TNFRSF9). Human 4-1BB is located at chromosome 1, with a full length of 255 amino acids, comprising a signal peptide at amino acid 17, an extracellular region at amino acid 169, a transmembrane region at amino acid 27 and an intracellular region at amino acid 42. Mouse 4-1BB is located at mouse chromosome 4, which has a sequence similarity of about 60% with human 4-1BB. CD137 (4-1BB) is an inducible costimulatory receptor expressed on activated CD4+ and CD8+ T cells, NKT, NK cells, DC, macrophages, eosinophils, neutrophils and mast cells as well as Tregs.

In the present application, the term “CD35”, also written as CD3 zeta, generally refers to an amino acid residue coming from the cytoplasmic domain of ζ chain, which is sufficient to functionally deliver the initial signal required for the T cell activation.

In the present application, the term “hinge region” generally refers to a section of region between scFv and the transmembrane domain in the CAR structure. A hinge region generally derives from IgG family, e.g., IgG1 and IgG4. Some may derive from IgD and CD8. The hinge region generally has a certain flexibility, which affects the spatial constraint between the CAR molecule and its specific target, thereby affecting the contact between CAR T cells and tumor cells.

In the present application, the term “signal peptide” as used herein generally refers to a propeptide in a form of protein precursor existing as an N-terminal peptide. The signal peptide functions to promote the translocation of expression polypeptide linked to the endoplasmic reticulum. The signal peptide is generally resected during this process. The signal peptide may be heterogenous or homogenous to organisms used to producing polypeptides.

In the present application, the term “promoter” generally refers to a section of deoxyribonucleic acid (DNA) sequence enabling the transcription of particular genes. The promoter may be identified by RNA polymerases, and starts to transcribe and synthesize RNA. In the synthesis of ribonucleic acid (RNA), the promoter may interact with transcription factors that regulate the transcription of genes, thereby controlling the initiation time of gene expression (transcription) and the degree of expression. A promoter includes a core promoter region and a regulation region, which is located in the regulation sequence controlling the gene expression, upstream of the initiation site of gene expression (5′ direction of DNA antisense chain), without compilation function itself. In accordance with the mode of action and the function, the promoter is classified into three types: constitutive promoters (maintaining persistent activity in most or all of the tissues), specific promoters (tissue specific or specific during development period) and inducible promoters (regulated by external chemical or physical signals).

In the present application, the term “isolated” generally refers to being obtained artificially from native states. If a certain “isolated” substance or ingredient appears in nature, it could be that its natural environment has changed, or the substance has been isolated from the natural environment, or both. For example, there is a certain nonisolated polynucleotide or polypeptide naturally occurring in a living animal, then the same polynucleotide or polypeptide of a high purity isolated from this natural state is called isolated. The term “isolated” does not exclude being mixed with artificial or synthesized substances, nor excluding the presence of other impure substances with no influences on the activity of the substance.

In the present application, the term “isolated nucleic acid molecule” generally refers to isolated nucleotide, deoxyribonucleotide or ribonucleotide of any length, or analogues thereof isolated from its natural environment or synthesized artificially.

In the present application, the term “vector” generally refers to a nucleic acid carrier into which a polynucleotide encoding a certain protein can be inserted and enables the expression of the protein. The vector can make the genetic elements it carries be expressed in a host cell by transforming, transducing or transfecting the host cell. For example, the vector includes: plasmid; phagemid; Cosmid; artificial chromosomes, such as yeast artificial chromosomes (YAC), bacterial artificial chromosomes (BAC) or P1-derived artificial chromosomes (PAC); phages, such as λ phages or M13 phages and animal viruses, and the like. The species of animal viruses used as vectors are retrovirus (including lentivirus), adenovirus, adeno-associated virus, herpes virus (e.g., herpes simplex virus), poxvirus, baculovirus, papilloma virus, papovavirus (e.g., SV40). A vector may contain various elements for controlling the expression, including promoter sequences, transcription initiation sequences, enhancer sequences, selective elements and reporter genes. In addition, the vector may also contain replication origins. The vector may also probably include ingredients that help its entry into cells, such as virion, lipidosome or protein coat, but not only these substances.

In the present application, the term “immune effector cell” generally refers to cells that participate in immune response, e.g., promote immune effector response. Examples of immune effector cells include T cells, e.g., T cells of α/β and T cells of γ/δ, B cells, natural killer (NK) cells, natural killer T (NKT) cells, mast cells and bone marrow-derived phagocytes.

In the present application, the term “composition” generally refers to the composition suitable for administering to patients and human patients. For example, the composition of the present application may include the immune effector cells of the present application, and optionally pharmaceutically acceptable adjuvants. In some embodiments, the acceptable components of the composition are nontoxic to recipients at the used dosage and concentration. The composition of the present application includes, but not limited to, liquid, frozen and lyophilized composition.

In the present application, the term “tumor” generally refers to neoplasms or solid lesions formed from abnormal cell growth. In the present application, the tumor may be a solid tumor or a blood tumor. For example, in the present application, the tumor may be a GPC3 positive tumor, wherein the GPC3 positive tumor may include hepatic carcinoma.

In the present application, the term “comprising” generally refers to including explicitly specified characteristics, but not excluding other elements.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below a specified value.

### Chimeric Antigen Receptor (CAR)

In one aspect, the present application provides a chimeric antigen receptor (CAR), which comprises a GPC3 binding domain, a transmembrane domain, a costimulatory domain and an intracellular signaling domain, the GPC3 binding domain comprises an antibody or a fragment thereof which specifically binding to GPC3, wherein the antibody comprises a heavy chain complementary determining region 1 (HCDR1), a heavy chain complementary determining region 2 (HCDR2) and a heavy chain complementary determining region 3 (HCDR3), the amino acid sequence of the HCDR1 is as set forth in SEQ ID NO: 19, the amino acid sequence of the HCDR2 is as set forth in SEQ ID NO: 20 and the amino acid sequence of the HCDR3 is as set forth in SEQ ID NO: 21.

For example, the antibody of the present application may also comprise a light chain complementary determining region 1 (LCDR1), a light chain complementary determining region 2 (LCDR2) and a light chain complementary determining region 3 (LCDR3), the amino acid sequence of the LCDR1 is as set forth in SEQ ID NO: 1, the amino acid sequence of the LCDR2 is as set forth in SEQ ID NO: 2 and the amino acid sequence of the LCDR3 is as set forth in SEQ ID NO: 3.

For example, the GPC3 is a human GPC3, the amino acid sequence of which is as set forth in SEQ ID NO: 56.

### CDR

In the present application, the HCDR1 may include an amino acid sequence as set forth in SEQ ID NO: 19.

X₁YX₂MH (SEQ ID NO: 19), wherein X₁ may be D or A, X₂ may be A or E.

In the present application, the HCDR1 may include an amino acid sequence as set forth in any one of SEQ ID NOs: 4 and 12.

In the present application, the HCDR2 may include an amino acid sequence as set forth in SEQ ID NO: 20.

X₁LX₂X₃X₄X₅GX₆X₇X₈YX₉X₁₀X₁₁X₁₂X₁₃G (SEQ ID NO: 20), wherein X₁ may be G or A, X₂ may be S or D, X₃ may be W or P, X₄ may be N or K, X₅ may be S or T, X₆ may be S or Q, X₇ may be I or T, X₈ may be G or A, X₉ may be A or S, X₁₀ may be D or Q, X₁₁ may be S or K, X₁₂ may be V or F, X₁₃ may be K or Q.

In the present application, the HCDR2 may include an amino acid sequence as set forth in any one of SEQ ID NOs: 5, 10 and 13.

In the present application, the HCDR3 may include an amino acid sequence as set forth in SEQ ID NO: 21.

X₁X₂X₃X₄X₅X₆X₇X₈X₉X₁₀X₁₁ (SEQ ID NO: 21), wherein X₁ may be D or T, X₂ may be H or R, X₃ may be Tor F, X₄ may be I or Y, X₅ may be G or S, X₆ may be V or Y, X₇ may be G or A, X₈ may be A, Y or H, X₉ may be F or a blank, X₁₀ may be D or a blank, X₁₁ may be I or a blank.

In the present application, the HCDR3 may include an amino acid sequence as set forth in any one of SEQ ID NOs: 6, 11 and 14.

For example, the HCDR1 of the present application may include an amino acid sequence as set forth in SEQ ID NO: 4, the HCDR2 may include an amino acid sequence as set forth in SEQ ID NO: 5, the HCDR3 may include an amino acid sequence as set forth in SEQ ID NO: 6.

For example, the HCDR1 of the present application may include an amino acid sequence as set forth in SEQ ID NO: 12, the HCDR2 may include an amino acid sequence as set forth in SEQ ID NO: 10, the HCDR3 may include an amino acid sequence as set forth in SEQ ID NO: 11.

For example, the HCDR1 of the present application may include an amino acid sequence as set forth in SEQ ID NO: 12, the HCDR2 may include an amino acid sequence as set forth in SEQ ID NO: 13, the HCDR3 may include an amino acid sequence as set forth in SEQ ID NO: 14.

In the present application, the LCDR1 may include an amino acid sequence as set forth in SEQ ID NO: 16.

X₁X₂X₃X₄SXSVX₆X₇X₈X₉YX₁₀X₁₁X₁₂X₁₃ (SEQ ID NO: 16), wherein X₁ may be T or R, X₂ may be G or S, X₃ may be T or S, X₄ may be S or Q, X₅ may be D or L, X₆ may be G or H, X₇ may be G or S, X₈ may be Y or N, X₉ may be N or G, X₁₀ may be V or T, X₁₁ may be S or Y, X₁₂ may be a blank or L, X₁₃ may be a blank or H.

In the present application, the LCDR1 may include an amino acid sequence as set forth in any one of SEQ ID NOs: 1 and 7.

In the present application, the LCDR2 may include an amino acid sequence as set forth in SEQ ID NO: 17.

X₁X₂SX₃RX₄S (SEQ ID NO: 17), wherein X₁ may be D or K, X₂ may be V or G, X₃ may be N, Y or Q, X₄ may be P or G.

In the present application, the LCDR2 may include an amino acid sequence as set forth in any one of SEQ ID NOs: 2, 8 and 15.

In the present application, the LCDR3 may include an amino acid sequence as set forth in SEQ ID NO: 18.

X₁X₂X₃X₄X₅X₆X₇X₈X₉X₁₀ (SEQ ID NO: 18), wherein X₁ may be S or G, X₂ may be S or Q, X₃ may be Y or S, X₄ may be A or G, X₅ may be S or L, X₆ may be G or T, X₇ may be S or P, X₈ may be T or P, X₉ may be L or T, X₁₀ may be V or a blank.

In the present application, the LCDR3 may include an amino acid sequence as set forth in any one of SEQ ID NOs: 3 and 9.

For example, the LCDR1 of the present application may include an amino acid sequence as set forth in SEQ ID NO: 1, the LCDR2 may include an amino acid sequence as set forth in SEQ ID NO: 2, the LCDR3 may include an amino acid sequence as set forth in SEQ ID NO: 3.

For example, the LCDR1 of the present application may include an amino acid sequence as set forth in SEQ ID NO: 7, the LCDR2 may include an amino acid sequence as set forth in SEQ ID NO: 8, the LCDR3 may include an amino acid sequence as set forth in SEQ ID NO:

9.

For example, the LCDR1 of the present application may include an amino acid sequence as set forth in SEQ ID NO: 7, LCDR2 may include an amino acid sequence as set forth in SEQ ID NO: 15, LCDR3 may include an amino acid sequence as set forth in SEQ ID NO: 9.

Further for example, the LCDR1 of the present application may include an amino acid sequence as shown in SEQ ID NO: 1, the LCDR2 may include an amino acid sequence as set forth in SEQ ID NO: 2, the LCDR3 may include an amino acid sequence as set forth in SEQ ID NO: 3, and the HCDR1 may include an amino acid sequence as set forth in SEQ ID NO: 4, the HCDR2 may include an amino acid sequence as set forth in SEQ ID NO: 5, the HCDR3 may include an amino acid sequence as set forth in SEQ ID NO: 6.

Further for example, the LCDR1 of the present application may include an amino acid sequence as shown in SEQ ID NO: 7, the LCDR2 may include an amino acid sequence as set forth in SEQ ID NO: 8, the LCDR3 may include an amino acid sequence as set forth in SEQ ID NO: 9, and the HCDR1 may include an amino acid sequence as set forth in SEQ ID NO: 12, the HCDR2 may include an amino acid sequence as set forth in SEQ ID NO: 10, the HCDR3 may include an amino acid sequence as set forth in SEQ ID NO: 11.

Further for example, the LCDR1 of the present application may include an amino acid sequence as set forth in SEQ ID NO: 7, the LCDR2 may include an amino acid sequence as set forth in SEQ ID NO:8, the LCDR3 may include an amino acid sequence as set forth in SEQ ID NO: 9, and the HCDR1 may include an amino acid sequence as set forth in SEQ ID NO: 12, the HCDR2 may include an amino acid sequence as set forth in SEQ ID NO: 13, the HCDR3 may include an amino acid sequence as set forth in SEQ ID NO: 14.

Further for example, the LCDR1 of the present application may include an amino acid sequence as set forth in SEQ ID NO: 7, the LCDR2 may include an amino acid sequence as set forth in SEQ ID NO: 15, the LCDR3 may include an amino acid sequence as set forth in SEQ ID NO: 9, and the HCDR1 may include an amino acid sequence as set forth in SEQ ID NO: 12, the HCDR2 may include an amino acid sequence as set forth in SEQ ID NO: 13, the HCDR3 may include an amino acid sequence as set forth in SEQ ID NO: 14.

### VL and VH

The chimeric antigen receptor of the present application may include a light chain variable region VL of the antibody and a heavy chain variable region VH of the antibody. For example, the VL may include an amino acid sequence as set forth in SEQ ID NO: 25, the VH may include an amino acid sequence as set forth in SEQ ID NO: 29. Further for example, the VL of the chimeric antigen receptor may include an amino acid sequence as set forth in any one of SEQ ID NOs: 22-24. The VH of the chimeric antigen receptor may include an amino acid sequence as set forth in any one of SEQ ID NOs: 26-28.

For example, the VL may include an amino acid sequence as set forth in SEQ ID NO: 22, the VH may include an amino acid sequence as set forth in SEQ ID NO: 26.

For example, the VL may include an amino acid sequence as set forth in SEQ ID NO: 23, the VH may include an amino acid sequence as set forth in SEQ ID NO: 27.

For example, the VL may include an amino acid sequence as set forth in SEQ ID NO: 23, the VH may include an amino acid sequence as set forth in SEQ ID NO: 28.

For example, the VL may include an amino acid sequence as set forth in SEQ ID NO: 24, the VH may include an amino acid sequence as set forth in SEQ ID NO: 28.

For example, in the present application, the chimeric antigen receptor may include HCDR1 in the VH of which the amino acid sequence is as set forth in SEQ ID NO: 29. For example, in the present application, the chimeric antigen receptor may include HCDR2 in the VH of which the amino acid sequence is as set forth in SEQ ID NO: 29. For example, in the present application, the chimeric antigen receptor may include HCDR3 in the VH of which the amino acid sequence is as set forth in SEQ ID NO: 29. Further for example, in the present application, the chimeric antigen receptor may include LCDR1 in the VL of which the amino acid sequence is as set forth in SEQ ID NO: 25. For example, in the present application, the chimeric antigen receptor may include LCDR2 in the VL of which the amino acid sequence is as set forth in SEQ ID NO: 25. For example, in the present application, the chimeric antigen receptor may include LCDR3 in the VL of which the amino acid sequence is as set forth in SEQ ID NO: 25.

### GPC3 Binding Domain

In the present application, the GPC3 binding domain of the chimeric antigen receptor may include the antibody specifically binding to GPC3 or an antigen-binding fragment thereof. For example, the antibody specifically binding to GPC3 or an antigen-binding fragment thereof in the present application may include, but not limited to, a recombinant antibody, a monoclonal antibody, a human antibody, a humanized antibody, a chimeric antibody, a bispecific antibody, a single-chain antibody, a double-antibody, a tri-antibody, a tetra-antibody, a Fv fragment, a scFv fragment, a Fab fragment, a Fab′ fragment, a F(ab′)2 fragment and a camelized single domain antibody.

In the present application, the antibody may be a humanized antibody. In other words, the antibody specifically binding to GPC3 or the antigen-binding fragment thereof in the present application may be an antibody immunological specifically binding to a related antigen (e.g., human GPC3) and comprising a complementary determining region (CDR) basically having a framework region (FR) of an amino acid sequence of a human antibody and basically having an amino acid sequence of a nonhuman antibody, or variants, derivatives, analogues or fragments thereof. “Basically” used herein, in the case of CDR, means that the amino acid sequence of CDR has an identity of at least 80%, at least 85%, at least 90%, at least 95%, at least 98% or at least 99% with the amino acid sequence of CDR in a nonhuman antibody. The humanized antibody may basically include all, at least one and generally two variable domains (Fab, Fab′, F (ab′)2, FabC, Fv), wherein all or basically all the CDR regions correspond to the CDR regions of a nonhuman immunoglobulin (i.e., the antibody) and all or basically all the framework regions are framework regions having the consensus sequence of a human immunoglobulin. In some embodiments, a humanized antibody contains at least the variable domains of a light chain and a heavy chain. In some embodiments, a humanized antibody contains only a humanized heavy chain. In certain embodiments, a humanized antibody contains only a light chain and/or a humanized variable domain of a humanized heavy chain.

In the present application, the antigen-binding fragment may include Fab, Fab′, F(ab)₂, Fv fragment, F(ab′)₂, scFv, di-scFv and/or dAb.

In the present application, the GPC3 binding domain is a single-chain antibody. For example, the GPC3 binding domain is scFv. The scFv may include a sequence as set forth in SEQ ID NO: 30. For example, the GPC3 binding domain may include a light chain variable region of the antibody, a linker peptide, a heavy chain variable region of the antibody.

In the present application, the light chain variable region of the antibody may include an amino acid sequence as set forth in SEQ ID NO: 25, the linker peptide may include an amino acid sequence as set forth in SEQ ID NO: 35, the heavy chain variable region of the antibody may include an amino acid sequence as set forth in SEQ ID NO: 29.

For example, the light chain variable region of the antibody may include an amino acid sequence as set forth in SEQ ID NO: 22, the linker peptide may include an amino acid sequence as set forth in SEQ ID NO: 35, the heavy chain variable region of the antibody may include an amino acid sequence as set forth in SEQ ID NO: 26.

For example, the light chain variable region of the antibody may include an amino acid sequence as set forth in SEQ ID NO: 23, the linker peptide may include an amino acid sequence as set forth in SEQ ID NO: 35, the heavy chain variable region of the antibody may include an amino acid sequence as set forth in SEQ ID NO: 27.

For example, the light chain variable region of the antibody may include an amino acid sequence as set forth in SEQ ID NO: 23, the linker peptide may include an amino acid sequence as set forth in SEQ ID NO: 35, the heavy chain variable region of the antibody may include an amino acid sequence as set forth in SEQ ID NO: 28.

For example, the light chain variable region of the antibody may include an amino acid sequence as set forth in SEQ ID NO: 24, the linker peptide may include an amino acid sequence as set forth in SEQ ID NO: 35, the heavy chain variable region of the antibody may include an amino acid sequence as set forth in SEQ ID NO: 28.

For example, the GPC3 binding domain may include an amino acid sequence as set forth in SEQ ID NO: 31.

For example, the GPC3 binding domain may include an amino acid sequence as set forth in SEQ ID NO: 32.

For example, the GPC3 binding domain may include an amino acid sequence as set forth in SEQ ID NO: 33.

For example, the GPC3 binding domain may include an amino acid sequence as set forth in SEQ ID NO: 34.

In the present application, the chimeric antigen receptor (CAR) may include an amino acid sequence as set forth in any one of SEQ ID NOs: 31-34.

### Transmembrane Domain, Costimulatory Domain and Intracellular Signaling Domain

In the present application, the transmembrane domain may include a transmembrane domain of a protein selected from: CD28, CD3e, CD45, CD4, CD5, CD8, CD9, CD16, CD22, CD33, CD37, CD64, CD80, CD86, CD134, CD137 and CD154. In some embodiments, the transmembrane domain may include an amino acid sequence as set forth in SEQ ID NO: 36.

In the present application, the costimulatory domain may include a costimulatory domain of a protein selected from: CD137, CD28, 4-1BB, OX-40 and ICOS. For example, the costimulatory domain may include an amino acid sequence as set forth in SEQ ID NO: 37.

In the present application, the intracellular signaling domain may include a signaling domain derived from CD35. For example, the intracellular signaling domain may include an amino acid sequence as set forth in SEQ ID NO: 38.

In the present application, the chimeric antigen receptor (CAR) may include a GPC3 binding domain, a transmembrane domain, a costimulatory domain, and an intracellular signaling domain in turn from N-terminal to C-terminal. For example, the GPC3 binding domain may include an amino acid sequence as set forth in any one of SEQ ID NOs: 31-34, the transmembrane domain may include an amino acid sequence as set forth in SEQ ID NO: 36, the costimulatory domain may include an amino acid sequence as set forth in SEQ ID NO: 37, the intracellular signaling domain may include an amino acid sequence as set forth in SEQ ID NO: 38.

For example, the chimeric antigen receptor (CAR) may include a GPC3 binding domain, a transmembrane domain, a costimulatory domain, and an intracellular signaling domain in turn from N-terminal to C-terminal. For example, the GPC3 binding domain may include an amino acid sequence as set forth in SEQ ID NO: 31, the transmembrane domain may include an amino acid sequence as set forth in SEQ ID NO: 36, the costimulatory domain may include an amino acid sequence as set forth in SEQ ID NO: 37, the intracellular signaling domain may include an amino acid sequence as set forth in SEQ ID NO: 38.

For example, the chimeric antigen receptor (CAR) may include a GPC3 binding domain, a transmembrane domain, a costimulatory domain, and an intracellular signaling domain in turn from N-terminal to C-terminal. For example, the GPC3 binding domain may include an amino acid sequence as set forth in SEQ ID NO: 32, the transmembrane domain may include an amino acid sequence as set forth in SEQ ID NO: 36, the costimulatory domain may include an amino acid sequence as set forth in SEQ ID NO: 37, the intracellular signaling domain may include an amino acid sequence as set forth in SEQ ID NO: 38.

For example, the chimeric antigen receptor (CAR) may include a GPC3 binding domain, a transmembrane domain, a costimulatory domain, and an intracellular signaling domain in turn from N-terminal to C-terminal. For example, the GPC3 binding domain may include an amino acid sequence as set forth in SEQ ID NO: 33, the transmembrane domain may include an amino acid sequence as set forth in SEQ ID NO: 36, the costimulatory domain may include an amino acid sequence as set forth in SEQ ID NO: 37, the intracellular signaling domain may include an amino acid sequence as set forth in SEQ ID NO: 38.

For example, the chimeric antigen receptor (CAR) may include a GPC3 binding domain, a transmembrane domain, a costimulatory domain, and an intracellular signaling domain in turn from N-terminal to C-terminal. For example, the GPC3 binding domain may include an amino acid sequence as set forth in SEQ ID NO: 34, the transmembrane domain may include an amino acid sequence as set forth in SEQ ID NO: 36, the costimulatory domain may include an amino acid sequence as set forth in SEQ ID NO: 37, the intracellular signaling domain may include an amino acid sequence as set forth in SEQ ID NO: 38.

In the present application, the chimeric antigen receptor (CAR) may also include a hinge region, which is linked to the GPC3 binding domain and the transmembrane domain. For example, the hinge region derives from IgG family, e.g., the hinge region derives from IgG1. For example, the hinge region derives from IgG4. For example, the hinge region derives from IgD. For example, the hinge region derives from CD8. For example, the hinge region may include an amino acid sequence as set forth in SEQ ID NO: 39.

For example, the chimeric antigen receptor (CAR) may include a GPC3 binding domain, a hinge region, a transmembrane domain, a costimulatory domain, and an intracellular signaling domain in turn from N-terminal to C-terminal. For example, the GPC3 binding domain may include an amino acid sequence set forth in any one of SEQ ID NOs: 31-34, the hinge region may include an amino acid sequence as set forth in SEQ ID NO: 39, the transmembrane domain may include an amino acid sequence as set forth in SEQ ID NO: 36, the costimulatory domain may include an amino acid sequence as set forth in SEQ ID NO: 37, the intracellular signaling domain may include an amino acid sequence as set forth in SEQ ID NO: 38.

In the present application, the chimeric antigen receptor (CAR) may also be linked to a signal peptide. For example, the signal peptide derives from CD8. For example, the signal peptide may include an amino acid sequence as set forth in SEQ ID NO: 40.

For example, the chimeric antigen receptor (CAR) may include a signal peptide, a GPC3 binding domain, a hinge region, a transmembrane domain, a costimulatory domain, and an intracellular signaling domain in turn from N-terminal to C-terminal. For example, the signal peptide may include an amino acid sequence as set forth in SEQ ID NO: 40, the GPC3 binding domain may include an amino acid sequence as set forth in any one of SEQ ID NOs: 31-34, the hinge region may include an amino acid sequence as set forth in SEQ ID NO: 39, the transmembrane domain may include an amino acid sequence as set forth in SEQ ID NO: 36, the costimulatory domain may include an amino acid sequence as set forth in SEQ ID NO: 37, the intracellular signaling domain may include an amino acid sequence as set forth in SEQ ID NO: 38.

For example, the chimeric antigen receptor (CAR) may include an amino acid sequence as set forth in SEQ ID NO: 66.

For example, the chimeric antigen receptor (CAR) may include an amino acid sequence as set forth in SEQ ID NO: 68.

For example, the chimeric antigen receptor (CAR) may include an amino acid sequence as set forth in SEQ ID NO: 70.

For example, the chimeric antigen receptor (CAR) may include an amino acid sequence as set forth in SEQ ID NO: 72.

In the present application, the CAR may also be linked to a promoter. For example, the promoter is the constitutive promoter. For example, the promoter is an elongation factor-1α (EF-1α) promoter. For example, the promoter may include a nucleotide sequence as set forth in SEQ ID NO: 41.

### Nucleic Acid Molecule, Vector, Cell, Preparation Method and Pharmaceutical Composition

In another aspect, the present application also provides one or more isolated nucleic acid molecules, which may encode the chimeric antigen receptor (CAR) of the present application. The one or more isolated nucleic acid molecules of the present application may be isolated nucleotides, deoxyribonucleotides or ribonucleotides of any length, or analogues isolated from the natural environment or synthesized artificially, but they may encode the chimeric antigen receptor (CAR) of the present application.

In another aspect, the present application also provides a vector, which may include the nucleic acid molecule of the present application. The vector can make the genetic elements it carries be expressed in a host cell by transforming, transducing or transfecting the host cell. For example, the vector may include: plasmid; phagemid; Cosmid; artificial chromosomes, such as yeast artificial chromosomes (YAC), bacterial artificial chromosomes (BAC) or P1-derived artificial chromosomes (PAC); phages, such as λ phages or M13 phages, and animal virus, and the like. The species of animal viruses used as vectors are retrovirus (including lentivirus), adenovirus, adeno-associated virus, herpes virus (e.g., herpes simplex virus), poxvirus, baculovirus, papilloma virus, papovavirus (e.g., SV40). Further for example, the vector may contain various elements for controlling the expression, including promoter sequences, transcription initiation sequences, enhancer sequences, selective elements and reporter genes. In addition, the vector may also contain replication origins. Moreover, the vector may also include ingredients that help its entry into cells, such as virion, lipidosome or protein coat, but not only these substances.

In another aspect, the present application also provides an immune effector cell, which may include the nucleic acid molecule of the present application or the vector of the present application. The cell may include progenies of a single cell. Because of natural, occasional or intentional mutations, the progeny may be not always completely the same as the original parent cell (in terms of the morphology of a total DNA complement or genetically). In some embodiments, the cell may also include cells transfected in vitro with the vector of the present application. In some embodiments, the cell may be mammal cells. In some embodiments, the immune effector cell includes T lymphocytes, e.g., T lymphocytes of α/β and T lymphocytes of γ/δ. In some embodiments, the immune effector cell may include natural killer (NK) cells, natural killer T (NKT) cells, mast cells and bone marrow-derived phagocytes.

In another aspect, the present application also provides a method of preparing the immune effector cell of the present application, which may include introducing the isolated nucleic acid molecule of the present application or the vector of the present application into the immune effector cell.

In another aspect, the present application also provides a composition, which may include the immune effector cell of the present application. In some embodiments, the composition may also include optionally pharmaceutically acceptable adjuvants.

In some embodiments, the acceptable components of the composition are nontoxic to recipients at the used dosage and concentration. The pharmaceutical composition of the present application includes, but not limited to, liquid, frozen and lyophilized composition.

In some embodiments, the pharmaceutically acceptable adjuvants may include any and all solvents, dispersion media, isotonic agents and absorption retarders compatible with the immune effector cell, which are generally safe, nontoxic and not biologically or otherwise undesirable.

In some embodiments, the composition may be administered parenterally, transdermally, intraperitoneally, intra-arterially, intrathecally and/or intranasally or directly injected into tissues. For example, the composition may be administered to patients or subjects through infusion or injection. In some embodiments, the administration of the pharmaceutical composition may be performed through different modes, for example, intravenous, intraperitoneal, subcutaneous, intramuscular, topical or intradermal administration. In some embodiments, the pharmaceutical composition may be administered unremittingly. The unremitted (or continuous) administration may be realized by a small pump system worn by the patient, so as to measure the therapeutic agent flowing into the patient, as described in

### Use and Application

In another aspect, the present application also provides a use of the chimeric antigen receptor of the present application, the nucleic acid molecule of the present application, the vector of the present application, the immune effector cell of the present application and/or the composition of the present application in preparing a medicament, the medicament may be used to treat a disease or disorder related to the expression of GPC3.

In the present application, the disease or disorder related to the expression of GPC3 is a cancer or a malignant tumor. For example, the disease or disorder related to the expression of GPC3 may include hepatic carcinoma.

In another aspect, the present application also provides a method of preventing, alleviating or treating tumors, which may include administering the immune effector cell of the present application to a subject in need thereof. In the present application, the administration may be performed through different modes, for example, intravenous, intratumoral, intraperitoneal, subcutaneous, intramuscular, topical or intradermal administration.

In another aspect, the chimeric antigen receptor of the present application, the nucleic acid molecule of the present application, the vector of the present application, the immune effector cell of the present application and/or the composition of the present application may be used to prevent, alleviate or treat tumors.

In the present application, the tumors may be solid tumors or blood tumors.

In the present application, the subject may include human and nonhuman animals. For example, the subject may include, but not limited to, cat, dog, horse, pig, cow, sheep, rabbit, mouse, rat, or monkey.

Without intend to be limited by any theories, the examples below are only for interpreting the chimeric antigen receptor, the preparation method and the use thereof in the present application, rather than limiting the inventive scope of the present application. The examples do not include detailed description of traditional methods, such as methods for constructing vectors and plasmids, methods for inserting genes encoding a protein into such vectors and plasmids or methods for introducing plasmids into a host cell. Such methods are well-known to persons with ordinary skills in the art, and have been described in many publications, including Sambrook, J., Fritsch, E. F. and Maniais, T. (1989) Molecular Cloning: A Laboratory Manual, 2nd edition, Cold spring Harbor Laboratory Press.

### EXAMPLES

### Example 1. Preparation of scFv Fragment

The nucleic acid sequence of the light chain variable region and the nucleic acid sequence of the heavy chain variable region of 204A antibody were linked through the nucleic acid sequence of a linker peptide, obtaining 204AscFv nucleic acid sequence and amino acid sequence; the nucleic acid sequences of L1H2, L1H6, L2H6 antibodies were treated in the same way, obtaining L1H2scFv nucleic acid sequence and amino acid sequence, L1H6scFv nucleic acid sequence and amino acid sequence, L2H6scFv nucleic acid sequence and amino acid sequence. All the above scFv (204AscFv, L1H2scFv, L1H6scFv, L2H6scFv) nucleic acid sequences were delivered to Nanjing GenScript Biotechnology Co., Ltd. for gene synthesis, and a nucleic acid sequence of SEQ ID NO: 57 was added at the 5′-end of the scFv nucleic acid sequence, a nucleic acid sequence of SEQ ID NO: 58 was added at the 3′-end of the scFv nucleic acid sequence for molecular construction (homologous recombination).

SEQ ID NO: 59, SEQ ID NO: 60, SEQ ID NO: 60, SEQ ID NO: 61 show the nucleic acid sequences of the light chain variable regions of 204A, L1H2, L1H6, L2H6 antibodies, respectively; SEQ ID NO: 62, SEQ ID NO: 63, SEQ ID NO: 64, SEQ ID NO: 64 show the nucleic acid sequences of the heavy chain variable regions of 204A, L1H2, L1H6, L2H6 antibodies, respectively; SEQ ID NO:46 or SEQ ID NO:47 shows the nucleic acid sequence of the linker peptide; SEQ ID NO: 35 shows the amino acid sequence of the linker peptide; SEQ ID NO:42, SEQ ID NO: 31 show the nucleic acid sequence and amino acid sequence of 204AscFv, respectively; SEQ ID NO:43, SEQ ID NO: 32 show the nucleic acid sequence and amino acid sequence of L1H2scFv, respectively; SEQ ID NO:44, SEQ ID NO: 33 show the nucleic acid sequence and amino acid sequence of L1H6scFv, respectively; SEQ ID NO:45, SEQ ID NO: 34 show the nucleic acid sequence and amino acid sequence of L2H6scFv, respectively.

### Example 2. Construction of CAR Plasmid

BBz platform plasmids (Origninal Energy Self-construction) were double digested with MluI and NheI endonucleases (purchased from NEB) to produce 7924 bp linear fragments, which were recovered by tapping and then mixed with the scFv fragments prepared in Example 1 at a ratio of 100 ng:30 ng, respectively (the volume not greater than 10 μl), then transformed with E. coli DH5α competent cells, heat-shocked for 45 s and then coated on an ampicillin resistance-containing LB solid medium, and incubated at 37° C. overnight. The scFv sequences were linked to the digested BBz platform plasmids in E. coli through the homologous arms (SEQ ID NOs: 57-58) at its front and back in a manner of homologous recombination. Monoclonal colonies were selected and shaken to extract the plasmids, which were sequenced to verify the validity of the plasmids. The linking sequence of elements in various parts of the CAR structure in the constructed complete plasmid as well as the sequence number in the present application were shown in Table 1 below.

TABLE 1
Linking sequence of various parts of the
chimeric antigen receptor in CAR plasmid

| Chimeric | Elemental sequence in CAR structural region: |
|---|---|
| antigen | signal peptide-extracellular binding |
| receptor | region-transmembrane region-intracellular |
| plasmid | signal region 1-intracellular signal region 2 |
| BBz platform | CD8 signal peptide-multiple cloning |
| plasmid | site-CD8-CD137-CD3zeta |
| 204A-BBz | CD8 signal peptide-204AscFv-CD8-CD137-CD3zeta |
| L1H2-BBz | CD8 signal peptide-L1H2scFv-CD8-CD137-CD3zeta |
| L1H6-BBz | CD8 signal peptide- L1H6scFv-CD8-CD137-CD3zeta |
| L2H6-BBz | CD8 signal peptide-L2H6scFv-CD8-CD137-CD3zeta |
| GC33-BBz | CD8 signal peptide-GC33scFv-CD8-CD137-CD3zeta |

Table 1 shows the linking sequence of various parts of the chimeric antigen receptor in the CAR plasmid obtained in this example, and the linking can also refer to the attached FIG. 2 in the specification.

SEQ ID NO: 48 shows the nucleic acid sequence of the CAR structural region of the BBz platform plasmid, SEQ ID NO: 49 shows the nucleic acid sequence of the signal peptide in the CAR structural region of the BBz platform plasmid, SEQ ID NO: 40 shows the amino acid sequence of the signal peptide in the CAR structural region of the BBz platform plasmid, SEQ ID NO: 50 shows the nucleic acid sequence of the hinge region in the CAR structural region of the BBz platform plasmid, SEQ ID NO: 39 shows the amino acid sequence of the hinge region in the CAR structural region of the BBz platform plasmid, SEQ ID NO: 51 shows the nucleic acid sequence of the transmembrane region in the CAR structural region of the BBz platform plasmid, SEQ ID NO: 36 shows the amino acid sequence of the transmembrane region in the CAR structural region of the BBz platform plasmid, SEQ ID NO: 52 shows the nucleic acid sequence of the intracellular CD137 costimulatory domain in the CAR structural region of the BBz platform plasmid, SEQ ID NO: 37 shows the amino acid sequence of the intracellular CD137 costimulatory domain in the CAR structural region of the BBz platform plasmid, SEQ ID NO: 53 shows the nucleic acid sequence of the intracellular signaling domain CD3ZETA in the CAR structural region of the BBz platform plasmid, SEQ ID NO: 38 shows the amino acid sequence of the intracellular signaling domain CD3ZETA in the CAR structural region of the BBz platform plasmid, SEQ ID NO: 65 shows the nucleic acid sequence of the CAR structural region of 204A-BBz plasmid, SEQ ID NO: 66 shows the amino acid sequence of the CAR structural region of 204A-BBz plasmid, SEQ ID NO: 67 shows the nucleic acid sequence of the CAR structural region of L1H2-BBz plasmid, SEQ ID NO: 68 shows the amino acid sequence of the CAR structural region of L1H2-BBz plasmid, SEQ ID NO: 69 shows the nucleic acid sequence of the CAR structural region of L1H6-BBz plasmid, SEQ ID NO:70 shows the amino acid sequence of the CAR structural region of L1H6-BBz plasmid, SEQ ID NO: 71 shows the nucleic acid sequence of the CAR structural region of L2H6-BBz plasmid, SEQ ID NO: 72 shows the amino acid sequence of the CAR structural region of L2H6-BBz plasmid, SEQ ID NO: 54 shows the nucleic acid sequence of the CAR structural region of GC33-BBz plasmid, SEQ ID NO: 55 shows the amino acid sequence of the CAR structural region of GC33-BBz plasmid.

### Example 3. Packaging of Virus

As an example, the vector system used to construct the lentiviral plasmid vector of the invention belongs to the third generation of lentiviral vector system, which contains three plasmids totally, i.e., a packaging plasmid psPAX2 encoding Gag-Pol protein and Rev protein (donated by a laboratory); a PMD2.G plasmid encoding envelope protein VSV-G (donated by a laboratory), and a core plasmid containing encoding target gene CAR constructed in the above Table 1 (i.e., 204A-BBz, L1H2-BBz, L1H6-BBz, L2H6-BBz or GC33-BBz). Based on the gene encoding CAR in the core plasmid of the BBz platform plasmid, the expression was regulated by an elongation factor-1α (EF-1α) promoter.

The packaging process of viruses was as below:
(1) Suspending 10e6 of 293T cells in 2 ml DMEM culture medium containing 10% FBS and plating in a single pore of a 6-well plate, and culturing overnight;
(2) Sucking away 1 ml culture medium, adding 1 ml Opti-MEM culture medium containing 4.5 ug packaging plasmid (psPAX2:PMD2.G:core plasmid=3:2:4) and 13 μl FuGENEHD transfection reagent (Promega Co.), mixing gently and then culturing in a CO₂ incubator at 37° C. for 12 hours;
(3) Removing the culture medium containing plasmids, washing with PBS (Hyclone Co.) once and then replacing with 2 ml DMEM culture medium containing 5% FBS, culturing for 30 hours;
(4) Collecting 1 ml virus supernatant, which was centrifuged at 3000 rpm for 5 minutes and stored at 4° C. temporarily, supplementing 1 ml fresh DMEM culture medium containing 5% FBS into the plate to continue culturing for 24 hours;
(5) Harvesting all the 2 ml virus supernatant, which was centrifuged at 3000 rpm for 5 minutes and mixed uniformly with the previously collected 1 ml supernatant, then packaged and cryopreserved at −80° C. for use.

SEQ ID NO: 41 shows the nucleic acid sequence of the elongation factor-1α.

### Example 4. Determination of Virus Titer

Virus titers were detected following the method below:
(1) Formulating DMEM complete culture medium containing 8 ug/ml polybrene;
(2) Taking 293T cells in good condition, removing the supernatant, washing with PBS and then digesting with 0.25% pancreatin (GIBICO) at 37° C. for 3 minutes, then resuspending with a small amount of the complete culture medium obtained in step (1) of this example and counting, and adjusting the cell concentration to 1×10e5 cells/ml with the complete culture medium obtained in step (1) of this example;
(3) Adding 1 ml cell suspension (1×10e5 cells) into each tube, then adding 2 μl virus solution resulting from example 3 and 4 μl double diluted virus solution respectively, mixing uniformly and adding into a 12-well plate, with 1 ml each well. Additionally adding 1 ml cell suspension containing no virus (1×10e5 cells) into 4 wells respectively, which were used as blank pairs in the detection by flow cytometry.
(4) Additionally adding 1 ml cell suspension containing no virus (1×10e5 cells) into 4 wells respectively, digesting the cells after adherence for 6 hours and then counting, taking the average as the final number of planking cells;
(5) Placing the cell culture plate in an incubator at 37° C., 5% CO₂ for stationary culture for 72 hours;
(6) Taking cells 72 hours after being inoculated with viruses, sucking away the culture medium in the wells carefully, adding 1 ml PBS into each well along the wall slowly, rinsing gently and then sucking away (Pay attention to preventing excessive falling off of cells);
(7) Removing the supernatant, washing with PBS and then digesting with 0.25% pancreatin (GIBICO) at 37° C., flapping the well plate gently and stopping the digestion when observing under a microscope that most cells have been not adhered to the wall;
(8) Adding 1 ml complete culture medium obtained in step (1) of this example into each well to stop the digestion, blowing the well wall gently with a pipetting gun until all the cells fell off, transferring into 1.5 ml sterile centrifugal tubes, and counting the cells;
(9) Taking an equal amount of cells (2˜5×10e5) from each tube, numbering (the tube number should be consistent with the number of the tube into which virus was added), supplementing to 1.2 ml with NBS solution (PBS solution containing 1% new-born calf serum (GIBICO)), centrifuging for 5 min at 4° C. and 2000 rpm, and discarding the supernatant:
(10) Adding 1 ml NBS into each tube, mixing uniformly by blowing gently, centrifuging for 5 min at 4° C. and 2000 rpm, and sucking away the supernatant fully;
(11) Adding 50 μl NBS and 0.5 μg CAR detection primary antibody into each tube, mixing uniformly by blowing gently with a pipetting gun (no addition into the blank well), and incubating at 4° C. for 60 min;
(12) Directly adding 1 ml NBS for resuspension, centrifuging for 5 min at 4° C. and 2000 rpm, and sucking away the supernatant;
(13) Adding 1 ml NBS for resuspension, centrifuging for 5 min at 4° C. and 2000 rpm, and sucking away the supernatant fully;
(14) Adding 50 μl NBS and 0.5 μl secondary antibody (the concentration of the secondary antibody was 1 mg/ml) (no secondary antibody was added into the blank well), and incubating at 4° C. for 30 min;
(15) Repeating steps (13)-(14);
(16) Adding 200 μl NBS into each tube for resuspension;
(17) Detecting fluorescence expression percentage on a BDFACSCantoII flow cytometer, the fluorescence was Dylight 650;
(18) Data process:

Virus titer=[Number of planking cells (Counting value of cells)×(Positive rate of test tube−Positive rate of the control tube)]/Volume of inoculated virus solution (converting into the volume before dilution)

Quality control point: When the positive rate of positive tube-the positive rate of the control tube was 5%-20%, the results were useable.

Upon detection, the titers of virus supernatant packages were shown in Table 2, the titers were in a range of 9-30×10e6/ml, and they can be directly used in T cell infection.

TABLE 2
Virus Titer

| Name of virus | Titer | |
|---|---|---|
| 204A-BBz | 1.325*10⁷ | IU/ml |
| L1H2-BBz | 9.1*10⁶ | IU/ml |
| L1H6-BBz | 1.31*10⁷ | IU/ml |
| L2H6-BBz | 1.0*10⁷ | IU/ml |
| GC33-BBz | 2.68*10⁷ | IU/ml |

### Example 5. Infection and Amplification of T Cells

### CAR-T Cells containing GPC3 antibody-based scFv sequences were produced as below:

(1) Obtaining human peripheral blood mononuclear cells by density gradient centrifugation (provided by the volunteers of Ark project of Shanghai Protocell Medical Technology Co., Ltd);

(2) Resuspending the peripheral blood mononuclear cells with X-VIVO (Lonza Co.) culture medium containing 200 U/ml of interleukin 2 (IL-2) to a cell density of 2×10⁶/m, and adding CD3/CD28 magnetic beads (Thermo Fisher) at a ratio of 1:3 to activate T cells;
(3) Statically culturing the activated peripheral blood mononuclear cells in an incubator at 37° C. and CO₂ for 24 hours;
(4) Adding the virus supernatant obtained from example 3 at a multiplicity of infection (MOI) of 5, adding polybrene to a final concentration of 5 ug/ml, placing the cell suspension in the well plate and centrifuging at 1200 rpm in a horizontal centrifuge for 1 hour;
(5) Returning the well plate to the 37° C./CO₂ incubator to culture for 24 hours;
(6) Centrifuging at 300 g for 5 minutes to remove the supernatant, resuspending the cells with a fresh X-VIVO culture medium containing 500 U/ml of interleukin 2 to a cell density of 0.6×10e6/ml, and culturing in the 37° C./CO₂ incubator;
(7) Counting the cells every two days, and supplementing the fresh X-VIVO culture medium containing 500 U/ml of interleukin 2 to adjust the cell density back to 0.6×10e6/ml;
(8) Detection of cell positive rate on CAR-T cells which have been cultured for 9-14 days: Viruses used to infect cells carry GFP, after infecting cells with viruses, the GFP positive rate was detected by a flow cytometer, thereby obtaining the positive rate of CAR expression; cells of which the positive rate>20% may be used to conduct tumor killing tests;

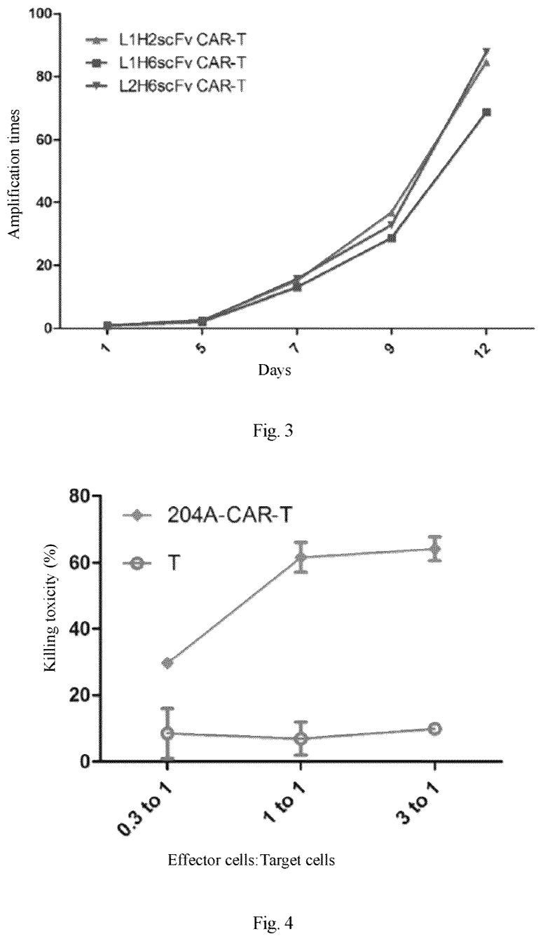

As shown in FIG. 2 and FIG. 3, the amplification times of T cells which have been activated and amplified for 9-12 days were between 20-100 folds, and the positive rates of infection were between 30% and 90% (Table 3), so they can be used in cytological function tests.

TABLE 3
Positive rate of CAR infection

| CAR Name | Positive rate of infection |
|---|---|
| 204A-BBz | 65.8% |
| L1H2-BBz | 79.1% |
| L1H6-BBz | 74.5% |
| L2H6-BBz | 80.6% |
| GC33-BBz | 82.7% |

### Example 6. Evaluation of CAR-T In Vitro Tumor Killing Activity Experiment

HepG2 is a hepatic carcinoma cell highly expressed by GPC3 protein, which is used as the positive target cell in this example. Cells infected with CAR-T viruses were used as effector cells, and uninfected T cells may be used as the control effector cells. The specific experimental process was as below:
(1) Detecting the infection efficiency of CAR, adjusting the proportion of infected CAR with uninfected T cells to be consistent in each group;
(2) Uniformly mixing the effector cells and target cells at a ratio of effector cells:target cells (effector/target ratio)=0.3:1, 1:1, 3:1 in 200 μl X-VIVO culture medium, the number of target cells was 1×10e4/well, as the experiment group;
(3) Wells only containing the same number of effector cells as the experiment group were used as the self-release background group of effector cells;
(4) Wells only containing the same number of target cells as the experiment group were used as the self-release background group of target cells;
(5) Culturing the cells obtained from steps (2)-(4) in the 37° C./CO₂ incubator for 18 hours;
(6) Adding 20 μl 10×lysis buffer (CytoTox96 nonradioactive cytotoxicity assay kit, provided by Promega Co.) into partial wells only containing target cells and reacting for 45 minutes, as the maximal release of target cells.
(7) Centrifuging the cell culture plates resulted from step (5) at 300 g for 5 minutes respectively, collecting 50 μl supernatant to detect the release amount of lactic dehydrogenase (LDH), with the detection method referring to the instruction of CytoTox96 nonradioactive cytotoxicity assay kit (Promega Co.). LDH is a kind of stable cytoplasmic enzyme, which may be released out during cell lysis, and the release mode is substantially the same as that of 51Cr in radioassay. The released LDH exists in the supernatant of the culture medium and can be detected through coupling enzyme reaction. During the enzyme reaction, LDH can convert a tetrazolium salt (INT) into red formazan, where the amount of the generated red product is in proportion to the number of lytic cells.
(8) The cell killing activity can be calculated by the following formula:

Killing toxicity %=100×(Experimental group−Self-release of effector cells−Self-release of target cells+Background value of the culture medium)/(Maximal release of target cells−Self-release of target cells)

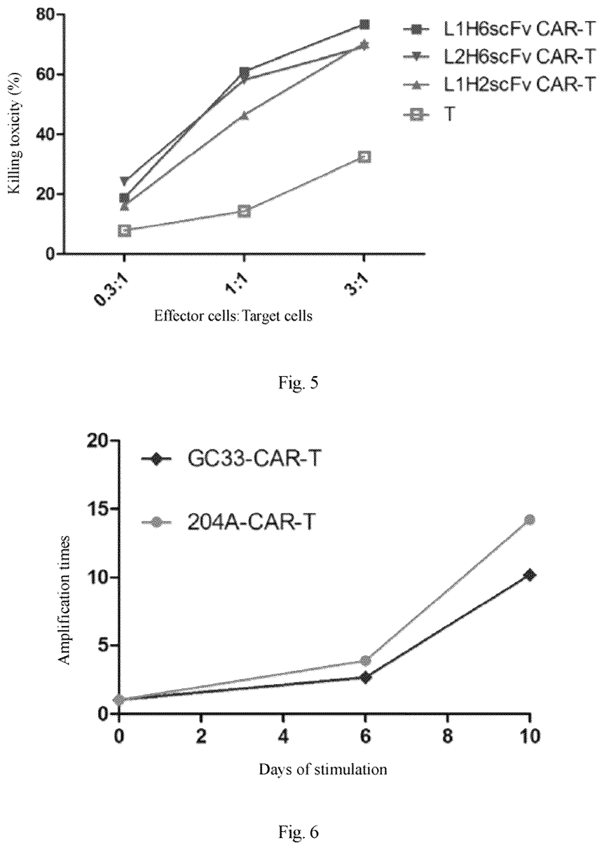

As shown in FIG. 4 and FIG. 5, compared to T cells not infected with viruses, all the 204A/L1H2/L1H6/L2H6 scFv-based CAR-T can effectively kill hepatic carcinoma target cells (HepG2 cells) in which GPC3 was highly expressed.

### Example 7. Evaluation of Cytological Function of CAR-T Based on CAR-T In Vitro Repetitive Stimulation Experiment

(1) CAR-T was prepared following the infection and amplification mode referred in Example 5; when it was amplified for 9-12 days, the positive rate was detected according to the method in Example 5; CAR-T cells were taken and resuspended to a density of 4×10e5/ml in a serum-free x-vivo 15 (lonza) culture medium, used as effector cells;
(2) Huh7 hepatic carcinoma cells in which GPC3 was moderately expressed were cultured in DMEM containing 10% serum and digested with pancreatin, the pancreatin reaction was terminated with the complete culture medium formulated in step (1) of Example 4, and then centrifuged at 1000 rpm for 5 minutes; after removing the supernatant, the cells were resuspended with 2 ml PBS, and then plated in a 10 cm petri dish that has been wetted, irradiated by ultraviolet-light for 10 minutes in a safety cabinet; the irradiated cells were washed, centrifuged and then resuspended to a density of 4×10e5/ml in a serum-free x-vivo 15 (lonza) culture medium, as the target cells;

500 μl effector cells were mixed with 500 μl target cells, and then cultured stationarily in the 37° C./5% CO₂ incubator; the colour of the culture medium was observed every two days, when the colour of the culture medium changed from orange to yellow, double volume of culture medium was supplemented.

Cells were counted on days 4 to 5 to calculate the amplification times.

After counting, 2×10e5 CAR-T effector cells were taken and mixed with 2×10e5 Huh7 cells that have been newly irradiated by ultraviolet-light again, and cultured stationarily in the 37° C./5% CO₂ incubator. The culture medium was observed every two days, and when the culture medium changed from orange to yellow; double volume of culture medium was supplemented.

After further culturing for 4 to 5 days, the cells were counted again to calculate the amplification times.

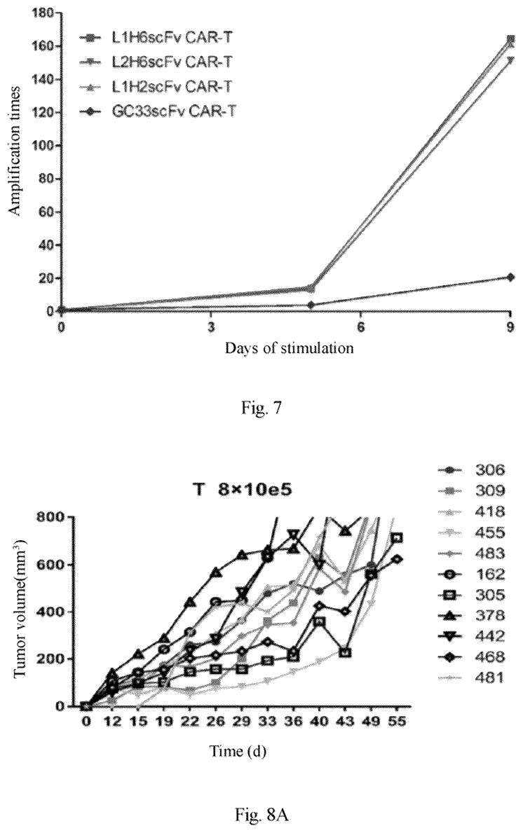

It can be seen from the repetitive stimulation results as shown in FIG. 6 and FIG. 7, the amplification times of 204A-CAR-T repetitive stimulation was higher than that of GC33-CAR-T, while the amplification times of L1H2/L1H6/L2H6-CAR-T through repetitive stimulation by Huh7 target cells was significantly higher than that of GC33scFv-based CAR-T, indicating that the clinical therapeutic effect of L1H2/L1H6/L2H6/204A-based CAR-T is superior to that of GC33-CAR-T.

### Example 8. CAR-T Tumor Inhibition Experiment (GPC3-Targeting)

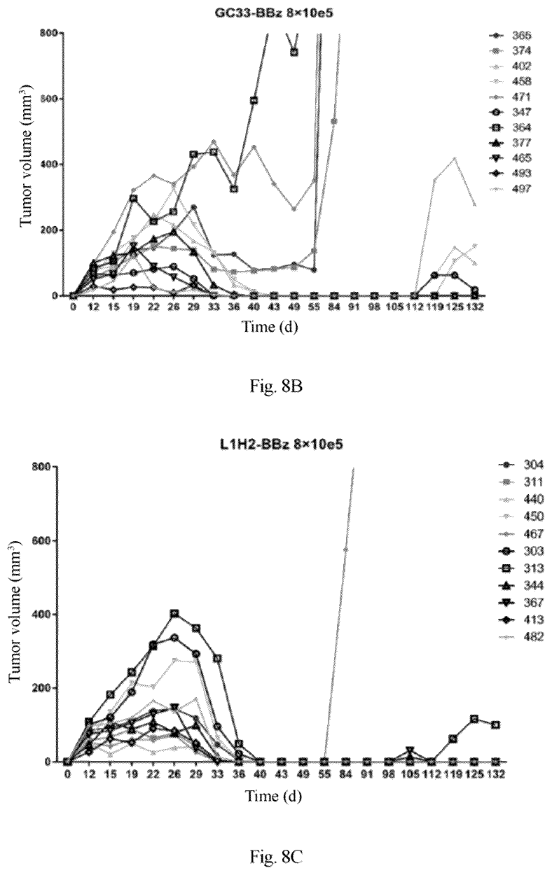

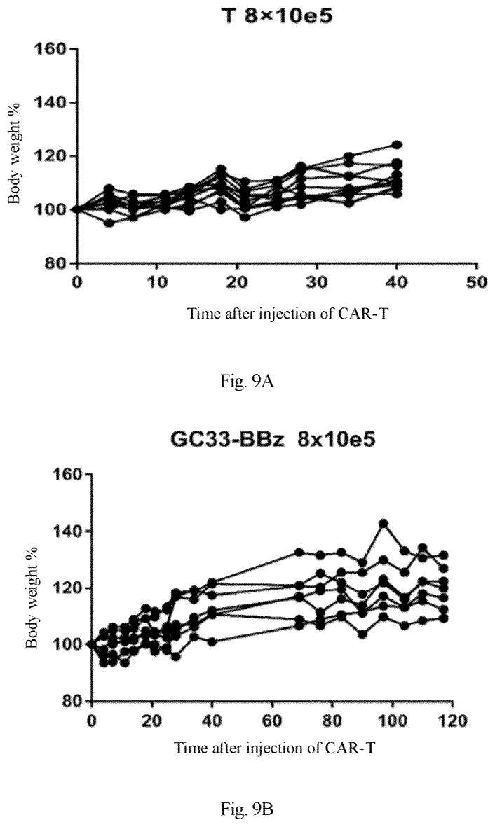

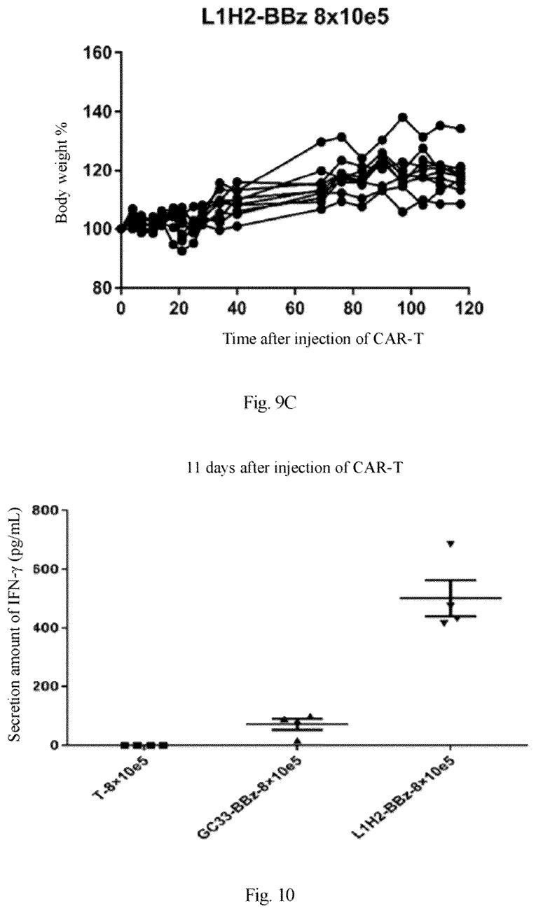

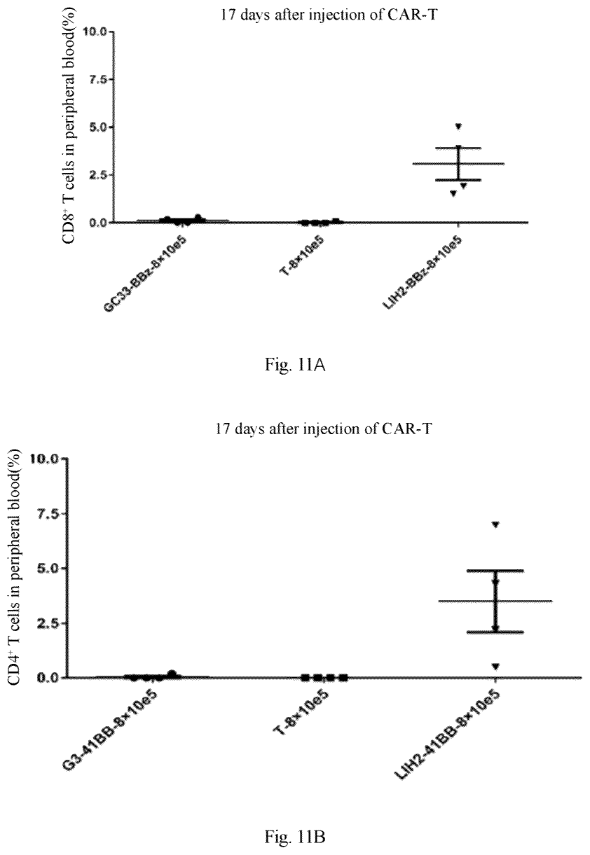

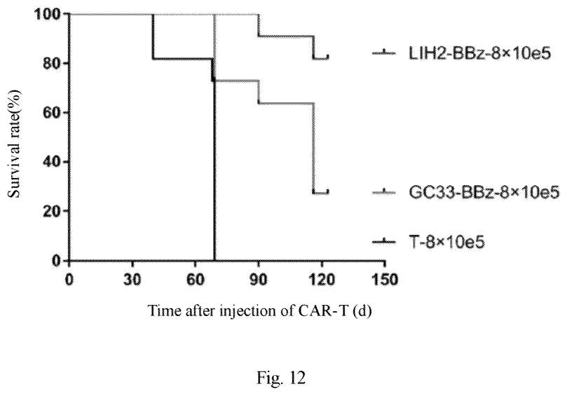

CAR-T cells expressing GC33-BBz, LIH2-41BBz as well as T cells uninfected with CAR-T were prepared following the method of Example 5. 6-week-old NSG mice subcutaneously burdening Huh7 tumor (1×10e7 each mouse) to a size of 80-150 mm³ were singly injected with CAR-T through tail vein at a dosage of 8×10e5 CAR positive cells, with 11 mice in each group. Body weight (FIGS. 9A-C) and tumor size (FIGS. 8A-C) were measured twice a week. On day 11 after injection of CAR-T, 4 mice were randomly selected from each group, from the tail vein of which anticoagulant blood was taken (heparin sodium anticoagulant) to detect IFNg in the blood (BD human th1/th2 CBA kit) (FIG. 10). On day 17 after injection of CAR-T, 4 mice were randomly selected from each group, from the tail vein of which anticoagulant blood was taken (heparin sodium anticoagulant) to detect human CD8 cell content (FIG. 11A) and human CD4 cell content (FIG. 11B) in blood by using flow cytometry. On day 85 after injection of CAR-T (98 days after tumor injection), the other side of back of mice without recurrence after elimination of tumor was burdened with tumor Huh7 for the second time ((1×10e7 for each mouse) to simulate the recurrence of tumor and observe the formation of the tumor (FIGS. 8A-C).

The experiment results showed that, compared with the GC33-BBz control group, the L1H2-BBz group had more significant effects of inhibiting and eliminating tumors as well as the capacity of resistance to recurrence (FIGS. 8A-8C), meanwhile, these groups did not show obvious toxic side effects (FIGS. 9A-9C, without obvious weight loss); the detection results of cytokines in vivo showed that the L1H2-BBz group had higher cytokine secretion, which was beneficial to promote the amplification of CAR-T in the body (FIG. 10); the detection results of in vivo cell proliferation showed that the LIH2-BBz group had higher in vivo cell proliferation capacity, which is positively related to the more superior effects of eliminating and inhibiting tumors (FIGS. 11A-11B). The survival curve of mice also showed that mice in the L1H2-BBz group had longer survival cycles than those in the GC33-BBz control group.

The foregoing detailed description is provided by means of explanations and examples, which is not intended to limit the scope of the attached claims. Various changes to the embodiments currently listed in the present application are apparent to persons with ordinary skills in the art, and are remained in the attached claims and its equivalent scope.

1. A chimeric antigen receptor (CAR), comprising:
a GPC3 binding domain;
a transmembrane domain;
a costimulatory domain; and
an intracellular signaling domain,
wherein the GPC3 binding domain comprises an antibody or the antigen-binding fragment thereof which specifically binds to GPC3,
wherein the antibody or the antigen-binding fragment thereof comprises:
a light chain complementary determining region 1 (LCDR1);
a light chain complementary determining region 2 (LCDR2);
a light chain complementary determining region 3 (LCDR3);
a heavy chain complementary determining region 1 (HCDR1);
a heavy chain complementary determining region 2 (HCDR2); and
a heavy chain complementary determining region 3 (HCDR3), and
wherein:
the amino acid sequence of the LCDR1 is as set forth in SEQ ID NO: 16,
the amino acid sequence of the LCDR2 is as set forth in SEQ ID NO: 17,
the amino acid sequence of the LCDR3 is as set forth in SEQ ID NO: 18,
the amino acid sequence of the HCDR1 is as set forth in SEQ ID NO: 19,
the amino acid sequence of the HCDR2 is as set forth in SEQ ID NO: 20, and
the amino acid sequence of the HCDR3 is as set forth in SEQ ID NO: 21.

2. The CAR of claim 1, wherein the LCDR1, LCDR2, LCDR3, HCDR1, HCDR2, and HCDR3 comprise the amino acid sequence selected from the group consisting of:
(1) the LCDR1 comprises the amino acid sequence as set forth in SEQ ID NO: 1,
the LCDR2 comprises the amino acid sequence as set forth in SEQ ID NO: 2,
the LCDR3 comprises the amino acid sequence as set forth in SEQ ID NO: 3,
the HCDR1 comprises the amino acid sequence as set forth in SEQ ID NO: 4.
the HCDR2 comprises the amino acid sequence as set forth in SEQ ID NO: 5, and
the HCDR3 comprises the amino acid sequence as set forth in SEQ ID NO: 6;
(2) the LCDR1 comprises the amino acid sequence as set forth in SEQ ID NO: 7,
the LCDR2 comprises the amino acid sequence as set forth in SEQ ID NO: 8,
the LCDR3 comprises the amino acid sequence as set forth in SEQ ID NO: 9,
the HCDR1 comprises the amino acid sequence as set forth in SEQ ID NO: 12,
the HCDR2 comprises the amino acid sequence as set forth in SEQ ID NO: 10, and
the HCDR3 comprises the amino acid sequence as set forth in SEQ ID NO: 11;
(3) the LCDR1 comprises the amino acid sequence as set forth in SEQ ID NO: 7,
the LCDR2 comprises the amino acid sequence as set forth in SEQ ID NO:8,
the LCDR3 comprises the amino acid sequence as set forth in SEQ ID NO: 9,
the HCDR1 comprises the amino acid sequence as set forth in SEQ ID NO: 12,
the HCDR2 comprises the amino acid sequence as set forth in SEQ ID NO: 13, and
the HCDR3 comprises the amino acid sequence as set forth in SEQ ID NO: 14; and
(4) the LCDR1 comprises the amino acid sequence as set forth in SEQ ID NO: 7,
the LCDR2 comprises the amino acid sequence as set forth in SEQ ID NO: 15,
the LCDR3 comprises the amino acid sequence as set forth in SEQ ID NO: 9,
the HCDR1 comprises the amino acid sequence as set forth in SEQ ID NO: 12,
the HCDR2 comprises the amino acid sequence as set forth in SEQ ID NO: 13, and
the HCDR3 comprises the amino acid sequence as set forth in SEQ ID NO: 14.

3. The CAR of claim 1, wherein:
the antibody or the antigen-binding fragment thereof further comprises a heavy chain variable region and a light chain variable region,
the amino acid sequence of the heavy chain variable region is as set forth in SEQ ID NO: 26, 27, or 28, and
the amino acid sequence of the light chain variable region is as set forth in SEQ ID NO: 22, 23, or 24.

4. (canceled)

5. The CAR of claim 1, wherein the antibody or the antigen-binding fragment thereof is a single-chain antibody.

6. The CAR of claim 1, wherein the antibody or the antigen-binding fragment thereof further comprises an amino acid sequence as set forth in SEQ ID NO: 31, 32, 33, or 34.

7. The CAR of claim 1, wherein the transmembrane domain comprises a transmembrane domain derived from a protein selected from: CD28, CD3e, CD45, CD4, CD5, CD8, CD9, CD16, CD22, CD33, CD37, CD64, CD80, CD86, CD134, CD137, and CD154.

8. The CAR of claim 1, wherein said the transmembrane domain comprises an amino acid sequence as set forth in SEQ ID NO: 36.

9. The CAR of claim 1, wherein the costimulatory domain comprises a costimulatory domain of a protein selected from: CD137, CD28, 4-1BB, OX-40 and ICOS.

10. The CAR of claim 1, wherein said the costimulatory domain comprises an amino acid sequence as shown in SEQ ID NO: 37.

11. The CAR of claim 1, wherein the intracellular signaling domain comprises a signaling domain derived from CD3ζ.

12. The CAR of claim 1, wherein the intracellular signaling domain comprises an amino acid sequence as set forth in SEQ ID NO: 38.

13. The CAR of claim 1, wherein the CAR further comprises a hinge region, which is linked to the GPC3 binding domain and the transmembrane domain.

14. (canceled)

15. The CAR of claim 1, wherein the CAR further comprises a linking signal peptide.

16-19. (canceled)

20. The CAR of claim 1, further comprising an amino acid sequence as set forth in any one of SEQ ID NOs: 31-34.

21. An isolated nucleic acid molecule, which encodes the CAR of claim 1.

22. (canceled)

23. A vector, which comprises the isolated nucleic acid molecule of claim 21.

24. (canceled)

25. An immune effector cell, which comprises the CAR claim 1.

26-27. (canceled)

28. A composition, which comprises the immune effector cell of claim 25.

29. A method for preventing or treating a disease or disorder, comprising administering the immune effector cell of claim 25 to a subject in need thereof, wherein the disease or disorder is related to the expression of GPC3.

30. The method of claim 29, wherein the disease or disorder is a GPC3-positive cancer or a malignant tumor.


SEQUENCE LISTING (72 sequences)

SEQ ID NO: 1 - protein, 14 aa, Artificial Sequence
  204A LCDR1
TGTSSDVGGYNYVS

SEQ ID NO: 2 - protein, 7 aa, Artificial Sequence
  204A LCDR2
DVSNRPS

SEQ ID NO: 3 - protein, 10 aa, Artificial Sequence
  204A LCDR3
SSYASGSTLV

SEQ ID NO: 4 - protein, 5 aa, Artificial Sequence
  204A HCDR1
DYAMH

SEQ ID NO: 5 - protein, 17 aa, Artificial Sequence
  204A HCDR2
GISWNSGSIGYADSVKG

SEQ ID NO: 6 - protein, 11 aa, Artificial Sequence
  204A HCDR3
DHTIGVGAFDI

SEQ ID NO: 7 - protein, 16 aa, Artificial Sequence
  L1H2/L1H6/L2H6 LCDR1
RSSQSLVHSNGYTYLH

SEQ ID NO: 8 - protein, 7 aa, Artificial Sequence
  L1H2/L1H6 LCDR2
KVSYRGS

SEQ ID NO: 9 - protein, 9 aa, Artificial Sequence
  L1H2/L1H6/L2H6 LCDR3
GQSGLTPPT

SEQ ID NO: 10 - protein, 17 aa, Artificial Sequence
  L1H2 HCDR2
ALDPKTGQTAYSQKFQG

SEQ ID NO: 11 - protein, 8 aa, Artificial Sequence
  L1H2 HCDR3
TRFYSYAY

SEQ ID NO: 12 - protein, 5 aa, Artificial Sequence
  L1H2/L1H6/L2H6 HCDR1
AYEMH

SEQ ID NO: 13 - protein, 17 aa, Artificial Sequence
  L1H6/L2H6 HCDR2
ALDPKSGSTAYSQKFQG

SEQ ID NO: 14 - protein, 8 aa, Artificial Sequence
  L1H6/L2H6 HCDR3
TRFYSYAH

SEQ ID NO: 15 - protein, 7 aa, Artificial Sequence
  L2H6 LCDR2
KGSQRPS

SEQ ID NO: 16 - protein, 16 aa, Artificial Sequence
  LCDR1 general formula
  X (1)..(1): X can be T or R
  X (2)..(2): X can be G or S
  X (3)..(3): X3 can be T or S
  X (4)..(4): X can be S or Q
  X (6)..(6): X can be D or L
  X (8)..(8): X can be G or H
  X (9)..(9): X can be G or S
  X (10)..(10): X can be Y or N
  X (11)..(11): X can be N or G
  X (13)..(13): X can be V or T
  X (14)..(14): X can be S or Y
  X (15)..(15): X can be L or does not exist
  X (16)..(16): X can be H or does not exist
XXXXSXVXXXXYXXXX

SEQ ID NO: 17 - protein, 7 aa, Artificial Sequence
  LCDR2 general formula
  X (1)..(1): X can be D or K
  X (2)..(2): X can be V or G
  X (4)..(4): X can be N, Y or Q
  X (6)..(6): X can be P or G
XXSXRXS

SEQ ID NO: 18 - protein, 10 aa, Artificial Sequence
  LCDR3 general formula
  X (1)..(1): X can be S or G
  X (2)..(2): X can be S or Q
  X (3)..(3): X can be Y or S
  X (4)..(4): X can be A or G
  X (5)..(5): X can be S or L
  X (6)..(6): X can be G or T
  X (7)..(7): X can be S or P
  X (8)..(8): X can be T or P
  X (9)..(9): X can be L or T
  X (10)..(10): X can be V or does not exist
XXXXXXXXXX

SEQ ID NO: 19 - protein, 5 aa, Artificial Sequence
  HCDR1 general formula
  X (1)..(1): X can be D or A
  X (3)..(3): X can be A or E
XYXMH

SEQ ID NO: 20 - protein, 17 aa, Artificial Sequence
  HCDR2 general formula
  X (1)..(1): X can be G or A
  X (3)..(3): X can be S or D
  X (4)..(4): X can be W or P
  X (5)..(5): X can be N or K
  X (6)..(6): X can be S or T
  X (8)..(8): X can be S or Q
  X (9)..(9): X can be I or T
  X (10)..(10): X can be G or A
  X (12)..(12): X can be A or S
  X (13)..(13): X can be D or Q
  X (14)..(14): X can be S or K
  X (15)..(15): X can be V or F
  X (16)..(16): X can be K or Q
XLXXXXGXXXYXXXXXG

SEQ ID NO: 21 - protein, 11 aa, Artificial Sequence
  HCDR3 general formula
  X (1)..(1): X can be D or T
  X (2)..(2): X can be H or R
  X (3)..(3): X can be T or F
  X (4)..(4): X can be I or Y
  X (5)..(5): X can be G or S
  X (6)..(6): X can be V or Y
  X (7)..(7): X can be G or A
  X (8)..(8): X can be A, Y or H
  X (9)..(9): X can be F or does not exist
  X (10)..(10): X can be D or does not exist
  X (11)..(11): X can be I or does not exist
XXXXXXXXXXX

SEQ ID NO: 22 - protein, 110 aa, Artificial Sequence
  204A VL
QSVLTQPASVSGSPGQSITISCTGTSSDVGGYNYVSWYQQHPGKAPKLMIYDVSNRPSGVSNRFSGSKSGNTASLTISGLQAEDEADYYCSSYASGSTLVFGGGTKLTVL

SEQ ID NO: 23 - protein, 112 aa, Artificial Sequence
  L1H2/L1H6 VL
DVVMTQSPLSLPVTLGQPASISCRSSQSLVHSNGYTYLHWYQQRPGQSPRLLIYKVSYRGSGVPDRFSGSGSGTDFTLKISRVEAEDVGVYYCGQSGLTPPTFGSGTKLEIK

SEQ ID NO: 24 - protein, 112 aa, Artificial Sequence
  L2H6 VL
DVVMTQSPLSLPVTLGQPASISCRSSQSLVHSNGYTYLHWYQQRPGQSPRLLIYKGSQRPSGVPDRFSGSGSGTDFTLKISRVEAEDVGVYYCGQSGLTPPTFGSGTKLEIK

SEQ ID NO: 25 - protein, 113 aa, Artificial Sequence
  VL general formula
  X (1)..(1): X can be Q or D
  X (2)..(2): X can be S or V
  X (4)..(4): X can be L or M
  X (7)..(7): X can be P or S
  X (8)..(8): X can be A or P
  X (9)..(9): X can be S or L
  X (10)..(10): X can be V or S
  X (11)..(11): X can be S or L
  X (12)..(12): X can be G or P
  X (13)..(13): X can be S or V
  X (14)..(14): X can be P or T
  X (15)..(15): X can be G or L
  X (16)..(16): X can be Q or G
  X (17)..(17): X can be S or Q
  X (18)..(18): X can be I or P
  X (19)..(19): X can be T or A
  X (20)..(20): X can be I or S
  X (21)..(21): X can be S or I
  X (22)..(22): X can be C or S
  X (23)..(23): X can be C or does not exist
  X (24)..(24): X can be T or R
  X (25)..(25): X can be G or S
  X (26)..(26): X can be T or S
  X (27)..(27): X can be S or Q
  X (29)..(29): X can be D or L
  X (31)..(31): X can be G or H
  X (32)..(32): X can be G or S
  X (33)..(33): X can be Y or N
  X (34)..(34): X can be N or G
  X (36)..(36): X can be V or T
  X (37)..(37): X can be S or Y
  X (38)..(38): X can be L or does not exist
  X (39)..(39): X can be H or does not exist
  X (44)..(44): X can be H or R
  X (47)..(47): X can be K or Q
  X (48)..(48): X can be A or S
  X (50)..(50): X can be K or R
  X (52)..(52): X can be M or L
  X (55)..(55): X can be D or K
  X (56)..(56): X can be V or G
  X (58)..(58): X can be N, Y or Q
  X (60)..(60): X can be P or G
  X (64)..(64): X can be S or P
  X (65)..(65): X can be N or D
  X (71)..(71): X can be K or G
  X (74)..(74): X can be N or T
  X (75)..(75): X can be T or D
  X (76)..(76): X can be A or F
  X (77)..(77): X can be S or T
  X (79)..(79): X can be T or K
  X (82)..(82): X can be G or R
  X (83)..(83): X can be L or V
  X (84)..(84): X can be Q or E
  X (88)..(88): X can be E or V
  X (89)..(89): X can be A or G
  X (90)..(90): X can be D or V
  X (94)..(94): X can be S or G
  X (95)..(95): X can be S or Q
  X (96)..(96): X can be Y or S
  X (97)..(97): X can be A or G
  X (98)..(98): X can be S or L
  X (99)..(99): X can be G or T
  X (100)..(100): X can be S or P
  X (101)..(101): X can be T or P
  X (102)..(102): X can be L or T
  X (103)..(103): X can be V or does not exist
  X (106)..(106): X can be G or S
  X (111)..(111): X can be T or E
  X (112)..(112): X can be V or I
  X (113)..(113): X can be L or K
XXVXTQXXXXXXXXXXXXXXXXXXXXXSXVXXXXYXXXXWYQQXPGXXPXLXIYXXSXRXSGVXXRFSGSXSGXXXXLXISXXXAEDXXXYYCXXXXXXXXXXFGXGTKLXXX

SEQ ID NO: 26 - protein, 120 aa, Artificial Sequence
  204A VH
EVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKDHTIGVGAFDIWGQGTMVTVSS

SEQ ID NO: 27 - protein, 115 aa, Artificial Sequence
  L1H2 VH
QVQLVQSGAEVKKPGASVKVSCKASGYDFTAYEMHWVRQAPGQGLEWMGALDPKTGQTAYSQKFQGRVTLTADKSISTAYMELSRLRSDDTVVYYCTRFYSYAYWGQGTLVTVSS

SEQ ID NO: 28 - protein, 115 aa, Artificial Sequence
  L1H6/L2H6 VH
QVQLVQSGAEVKKPGASVKVSCKASGYDFTAYEMHWVRQAPGQGLEWMGALDPKSGSTAYSQKFQGRVTLTADKSISTAYMELSRLRSDDTVVYYCTRFYSYAHWGQGTLVTVSS

SEQ ID NO: 29 - protein, 120 aa, Artificial Sequence
  VH general formula
  X (1)..(1): X can be E or Q
  X (9)..(9): X can be G or A
  X (10)..(10): X can be G or E
  X (11)..(11): X can be L or V
  X (12)..(12): X can be V or K
  X (13)..(13): X can be Q or K
  X (16)..(16): X can be R or A
  X (18)..(18): X can be L or V
  X (19)..(19): X can be R or K
  X (20)..(20): X can be L or V
  X (23)..(23): X can be A or K
  X (27)..(27): X can be F or Y
  X (28)..(28): X can be T or D
  X (30)..(30): X can be D or T
  X (31)..(31): X can be D or A
  X (33)..(33): X can be A or E
  X (43)..(43): X can be K or Q
  X (48)..(48): X can be V or M
  X (49)..(49): X can be S or G
  X (50)..(50): X can be G or A
  X (52)..(52): X can be S or D
  X (53)..(53): X can be W or P
  X (54)..(54): X can be N or K
  X (55)..(55): X can be S or T
  X (57)..(57): X can be S or Q
  X (58)..(58): X can be I or T
  X (59)..(59): X can be G or A
  X (61)..(61): X can be A or S
  X (62)..(62): X can be D or Q
  X (63)..(63): X can be S or K
  X (64)..(64): X can be V or F
  X (65)..(65): X can be K or Q
  X (68)..(68): X can be F or V
  X (70)..(70): X can be I or L
  X (71)..(71): X can be S or T
  X (72)..(72): X can be R or A
  X (74)..(74): X can be N or K
  X (75)..(75): X can be A or S
  X (76)..(76): X can be K or I
  X (77)..(77): X can be N or S
  X (78)..(78): X can be S or T
  X (79)..(79): X can be L or A
  X (81)..(81): X can be L or M
  X (82)..(82): X can be Q or E
  X (83)..(83): X can be M or L
  X (84)..(84): X can be N or S
  X (85)..(85): X can be S or R
  X (88)..(88): X can be A or S
  X (89)..(89): X can be E or D
  X (92)..(92): X can be A or V
  X (93)..(93): X can be L or V
  X (97)..(97): X can be A or does not exist
  X (98)..(98): X can be K or does not exist
  X (99)..(99): X can be D or T
  X (100)..(100): X can be H or R
  X (101)..(101): X can be T or F
  X (102)..(102): X can be I or Y
  X (103)..(103): X can be G or S
  X (104)..(104): X can be V or Y
  X (105)..(105): X can be G or A
  X (106)..(106): X can be A, Y or H
  X (107)..(107): X can be F or does not exist
  X (108)..(108): X can be D or does not exist
  X (109)..(109): X can be I or does not exist
  X (115)..(115): X can be M or L
XVQLVQSGXXXXXPGXSXXXSCXASGXXFXXYXMHWVRQAPGXGLEWXXXLXXXXGXXXYXXXXXGRXTXXXDXXXXXXYXXXXXLRXXDTXXYYCXXXXXXXXXXXXXWGQGTXVTVSS

SEQ ID NO: 30 - protein, 249 aa, Artificial Sequence
  ScFv general formula
  X (1)..(1): X can be Q or D
  X (2)..(2): X can be S or V
  X (3)..(3): X can be L or M
  X (4)..(4): X can be P or S
  X (5)..(5): X can be A or P
  X (6)..(6): X can be S or L
  X (7)..(7): X can be V or S
  X (8)..(8): X can be S or L
  X (9)..(9): X can be G or P
  X (10)..(10): X can be S or V
  X (11)..(11): X can be P or T
  X (12)..(12): X can be G or L
  X (13)..(13): X can be Q or G
  X (14)..(14): X can be S or Q
  X (15)..(15): X can be I or P
  X (16)..(16): X can be T or A
  X (17)..(17): X can be I or S
  X (18)..(18): X can be S or I
  X (19)..(19): X can be C or S
  X (20)..(20): X can be C or does not exist
  X (21)..(21): X can be T or R
  X (22)..(22): X can be G or S
  X (23)..(23): X can be T or S
  X (24)..(24): X can be S or Q
  X (25)..(25): X can be D or L
  X (26)..(26): X can be G or H
  X (27)..(27): X can be G or S
  X (28)..(28): X can be Y or N
  X (29)..(29): X can be N or G
  X (30)..(30): X can be V or T
  X (31)..(31): X can be S or Y
  X (32)..(32): X can be L or does not exist
  X (33)..(33): X can be H or does not exist
  X (34)..(34): X can be H or R
  X (35)..(35): X can be K or Q
  X (36)..(36): X can be A or S
  X (37)..(37): X can be K or R
  X (38)..(38): X can be M or L
  X (39)..(39): X can be D or K
  X (40)..(40): X can be V or G
  X (41)..(41): X can be N, Y or Q
  X (42)..(42): X can be P or G
  X (43)..(43): X can be S or P
  X (44)..(44): X can be N or D
  X (45)..(45): X can be K or G
  X (46)..(46): X can be N or T
  X (47)..(47): X can be T or D
  X (48)..(48): X can be A or F
  X (49)..(49): X can be S or T
  X (50)..(50): X can be T or K
  X (51)..(51): X can be G or R
  X (52)..(52): X can be L or V
  X (53)..(53): X can be Q or E
  X (54)..(54): X can be E or V
  X (55)..(55): X can be A or G
  X (56)..(56): X can be D or V
  X (57)..(57): X can be S or G
  X (58)..(58): X can be S or Q
  X (59)..(59): X can be Y or S
  X (60)..(60): X can be A or G
  X (61)..(61): X can be S or L
  X (62)..(62): X can be G or T
  X (63)..(63): X can be S or P
  X (64)..(64): X can be T or P
  X (65)..(65): X can be L or T
  X (66)..(66): X can be V or does not exist
  X (67)..(67): X can be G or S
  X (68)..(68): X can be T or E
  X (69)..(69): X can be V or I
  X (70)..(70): X can be L or K
  X (71)..(71): X can be G or does not exist
  X (72)..(72): X can be E or Q
  X (73)..(73): X can be G or A
  X (74)..(74): X can be G or E
  X (75)..(75): X can be L or V
  X (76)..(76): X can be V or K
  X (77)..(77): X can be Q or K
  X (78)..(78): X can be R or A
  X (79)..(79): X can be L or V
  X (80)..(80): X can be R or K
  X (81)..(81): X can be L or V
  X (82)..(82): X can be A or K
  X (83)..(83): X can be F or Y
  X (84)..(84): X can be T or D
  X (85)..(85): X can be D or T
  X (86)..(86): X can be D or A
  X (87)..(87): X can be A or E
  X (88)..(88): X can be K or Q
  X (89)..(89): X can be V or M
  X (90)..(90): X can be S or G
  X (91)..(91): X can be G or A
  X (92)..(92): X can be S or D
  X (93)..(93): X can be W or P
  X (94)..(94): X can be N or K
  X (95)..(95): X can be S or T
  X (96)..(96): X can be S or Q
  X (97)..(97): X can be I or T
  X (98)..(98): X can be G or A
  X (99)..(99): X can be A or S
  X (100)..(100): X can be D or Q
  X (101)..(101): X can be S or K
  X (102)..(102): X can be V or F
  X (103)..(103): X can be K or Q
  X (104)..(104): X can be F or V
  X (105)..(105): X can be I or L
  X (106)..(106): X can be S or T
  X (107)..(107): X can be R or A
  X (108)..(108): X can be N or K
  X (109)..(109): X can be A or S
  X (110)..(110): X can be K or I
  X (111)..(111): X can be N or S
  X (112)..(112): X can be S or T
  X (113)..(113): X can be L or A
  X (114)..(114): X can be L or M
  X (115)..(115): X can be Q or E
  X (116)..(116): X can be M or L
  X (117)..(117): X can be N or S
  X (118)..(118): X can be S or R
  X (119)..(119): X can be A or S
  X (120)..(120): X can be E or D
  X (121)..(121): X can be A or V
  X (122)..(122): X can be L or V
  X (123)..(123): X can be A or does not exist
  X (124)..(124): X can be K or does not exist
  X (125)..(125): X can be D or T
  X (126)..(126): X can be H or R
  X (127)..(127): X can be T or F
  X (128)..(128): X can be I or Y
  X (129)..(129): X can be G or S
  X (130)..(130): X can be V or Y
  X (131)..(131): X can be G or A
  X (132)..(132): X can be A, Y or H
  X (133)..(133): X can be F or does not exist
  X (134)..(134): X can be D or does not exist
  X (135)..(135): X can be I or does not exist
  X (136)..(136): X can be M or L
  misc_feature (138)..(142): Xaa can be any natural amino acid
  misc_feature (145)..(145): Xaa can be any natural amino acid
  misc_feature (147)..(149): Xaa can be any natural amino acid
  misc_feature (152)..(152): Xaa can be any natural amino acid
  misc_feature (156)..(157): Xaa can be any natural amino acid
  misc_feature (159)..(160): Xaa can be any natural amino acid
  misc_feature (162)..(162): Xaa can be any natural amino acid
  misc_feature (172)..(172): Xaa can be any natural amino acid
  misc_feature (177)..(179): Xaa can be any natural amino acid
  misc_feature (181)..(184): Xaa can be any natural amino acid
  misc_feature (186)..(188): Xaa can be any natural amino acid
  misc_feature (190)..(194): Xaa can be any natural amino acid
  misc_feature (197)..(197): Xaa can be any natural amino acid
  misc_feature (199)..(201): Xaa can be any natural amino acid
  misc_feature (203)..(208): Xaa can be any natural amino acid
  misc_feature (210)..(214): Xaa can be any natural amino acid
  misc_feature (217)..(218): Xaa can be any natural amino acid
  misc_feature (221)..(222): Xaa can be any natural amino acid
  misc_feature (226)..(238): Xaa can be any natural amino acid
  misc_feature (244)..(244): Xaa can be any natural amino acid
XXVXTQXXXXXXXXXXXXXXXXXXXXXSXVXXXXYXXXXWYQQXPGXXPXLXIYXXSXRXSGVXXRFSGSXSGXXXXLXISXXXAEDXXXYYCXXXXXXXXXXFGXGTKLXXXXGGGGSGGGGSGGGGSXVQLVQSGXXXXXPGXSXXXSCXASGXXFXXYXMHWVRQAPGXGLEWXXXLXXXXGXXXYXXXXXGRXTXXXDXXXXXXYXXXXXLRXXDTXXYYCXXXXXXXXXXXXXWGQGTXVTVSS

SEQ ID NO: 31 - protein, 245 aa, Artificial Sequence
  204AscFv
QSVLTQPASVSGSPGQSITISCTGTSSDVGGYNYVSWYQQHPGKAPKLMIYDVSNRPSGVSNRFSGSKSGNTASLTISGLQAEDEADYYCSSYASGSTLVFGGGTKLTVLGGGGSGGGGSGGGGSEVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKDHTIGVGAFDIWGQGTMVTVSS

SEQ ID NO: 32 - protein, 242 aa, Artificial Sequence
  L1H2scFv
DVVMTQSPLSLPVTLGQPASISCRSSQSLVHSNGYTYLHWYQQRPGQSPRLLIYKVSYRGSGVPDRFSGSGSGTDFTLKISRVEAEDVGVYYCGQSGLTPPTFGSGTKLEIKGGGGSGGGGSGGGGSQVQLVQSGAEVKKPGASVKVSCKASGYDFTAYEMHWVRQAPGQGLEWMGALDPKTGQTAYSQKFQGRVTLTADKSISTAYMELSRLRSDDTVVYYCTRFYSYAYWGQGTLVTVSS

SEQ ID NO: 33 - protein, 242 aa, Artificial Sequence
  L1H6scFv
DVVMTQSPLSLPVTLGQPASISCRSSQSLVHSNGYTYLHWYQQRPGQSPRLLIYKVSYRGSGVPDRFSGSGSGTDFTLKISRVEAEDVGVYYCGQSGLTPPTFGSGTKLEIKGGGGSGGGGSGGGGSQVQLVQSGAEVKKPGASVKVSCKASGYDFTAYEMHWVRQAPGQGLEWMGALDPKSGSTAYSQKFQGRVTLTADKSISTAYMELSRLRSDDTVVYYCTRFYSYAHWGQGTLVTVSS

SEQ ID NO: 34 - protein, 242 aa, Artificial Sequence
  L2H6scFv
DVVMTQSPLSLPVTLGQPASISCRSSQSLVHSNGYTYLHWYQQRPGQSPRLLIYKGSQRPSGVPDRFSGSGSGTDFTLKISRVEAEDVGVYYCGQSGLTPPTFGSGTKLEIKGGGGSGGGGSGGGGSQVQLVQSGAEVKKPGASVKVSCKASGYDFTAYEMHWVRQAPGQGLEWMGALDPKSGSTAYSQKFQGRVTLTADKSISTAYMELSRLRSDDTVVYYCTRFYSYAHWGQGTLVTVSS

SEQ ID NO: 35 - protein, 15 aa, Artificial Sequence
  linker
GGGGSGGGGSGGGGS

SEQ ID NO: 36 - protein, 24 aa, Artificial Sequence
  transmembrane domain
IYIWAPLAGTCGVLLLSLVITLYC

SEQ ID NO: 37 - protein, 42 aa, Artificial Sequence
  costimulatory domain
KRGRKKLLYIFKQPFMRPVQTTQEEDGCSCRFPEEEEGGCEL

SEQ ID NO: 38 - protein, 112 aa, Artificial Sequence
  intracellular signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 45 aa, Artificial Sequence
  hinge region
TTTPAPRPPTPAPTIASQPLSLRPEACRPAAGGAVHTRGLDFACD

SEQ ID NO: 40 - protein, 21 aa, Artificial Sequence
  signal peptide
MALPVTALLLPLALLLHAARP

SEQ ID NO: 41 - DNA, 1197 bp, Artificial Sequence
  promoter
gtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccgcggatccgccacc

SEQ ID NO: 42 - DNA, 735 bp, Artificial Sequence
  encoding 204AscFv
cagtctgtgctgacgcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatacgcgagcggcagcacactggtattcggcggagggaccaagctcaccgtcctaggaggaggaggcagcggcggaggaggctccggcggcggcggctctgaggtgcagctggtacagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatcatacgattggggttggggcttttgatatctggggccaagggacaatggtcactgtctcttca

SEQ ID NO: 43 - DNA, 726 bp, Artificial Sequence
  encoding L1H2scFv
gacgtggtgatgacccagtcccctctgtctttacccgttacactgggccagcccgcttccatcagctgcagaagcagccagtctttagtgcacagcaacggctacacctatttacactggtaccagcagagacccggccagagccccagactgctgatctacaaggtgtcataccgtggatccggcgtgcccgataggtttagcggcagcggtagcggcaccgatttcactttaaagatctctcgtgtcgaggccgaggatgtgggcgtttactactgcggccagagcggcctcaccccccctaccttcggctccggcaccaagctggagatcaaaggaggcggaggatctggaggaggagggtctggaggcggcgggagccaagtgcagctggtgcagagcggcgccgaggtgaagaaacccggagccagcgtgaaggtgagctgtaaggccagcggctatgacttcaccgcctacgagatgcactgggtgagacaagctcccggtcaaggtctcgagtggatgggcgctttagatcccaagaccgggcagaccgcctactcccagaagttccaaggtcgtgtgactttaaccgccgacaagagcatctccaccgcctacatggagctgtctcgtctgaggagcgacgataccgtggtgtactattgcactcgtttttactcctacgcctactggggccaaggtacactggtgaccgtgagctcc

SEQ ID NO: 44 - DNA, 726 bp, Artificial Sequence
  encoding L1H6scFv
gacgtggtgatgacccagtcccctctgtctttacccgttacactgggccagcccgcttccatcagctgcagaagcagccagtctttagtgcacagcaacggctacacctatttacactggtaccagcagagacccggccagagccccagactgctgatctacaaggtgtcataccgtggatccggcgtgcccgataggtttagcggcagcggtagcggcaccgatttcactttaaagatctctcgtgtcgaggccgaggatgtgggcgtttactactgcggccagagcggcctcaccccccctaccttcggctccggcaccaagctggagatcaaaggaggcggaggatctggaggaggagggtctggaggcggcgggagccaagtgcagctggtgcagagcggcgccgaggtgaagaaacccggagccagcgtgaaggtgagctgtaaggccagcggctatgacttcaccgcctacgagatgcactgggtgagacaagctcccggtcaaggtctcgagtggatgggcgctttagatcccaagtccggttctaccgcctactcccagaagttccaaggtcgtgtgactttaaccgccgacaagagcatctccaccgcctacatggagctgtctcgtctgaggagcgacgataccgtggtgtactattgcactcgtttctactcatacgcccactggggccaaggtacactggtgaccgtgagctcc

SEQ ID NO: 45 - DNA, 726 bp, Artificial Sequence
  encoding L2H6scFv
gacgtggtgatgacccagtcccctctgtctttacccgttacactgggccagcccgcttccatcagctgcagaagcagccagtctttagtgcacagcaacggctacacctatttacactggtaccagcagagacccggccagagccccagactgctgatctacaaggggtcccagcgtccctccggcgtgcccgataggtttagcggcagcggtagcggcaccgatttcactttaaagatctctcgtgtcgaggccgaggatgtgggcgtttactactgcggccagagcggcctcaccccccctaccttcggctccggcaccaagctggagatcaaaggaggcggaggatctggaggaggagggtctggaggcggcgggagccaagtgcagctggtgcagagcggcgccgaggtgaagaaacccggagccagcgtgaaggtgagctgtaaggccagcggctatgacttcaccgcctacgagatgcactgggtgagacaagctcccggtcaaggtctcgagtggatgggcgctttagatcccaagtccggttctaccgcctactcccagaagttccaaggtcgtgtgactttaaccgccgacaagagcatctccaccgcctacatggagctgtctcgtctgaggagcgacgataccgtggtgtactattgcactcgtttctactcatacgcccactggggccaaggtacactggtgaccgtgagctcc

SEQ ID NO: 46 - DNA, 45 bp, Artificial Sequence
  encoding linker
ggaggaggaggcagcggcggaggaggctccggcggcggcggctct

SEQ ID NO: 47 - DNA, 45 bp, Artificial Sequence
  encoding linker 2
ggaggcggaggatctggaggaggagggtctggaggcggcgggagc

SEQ ID NO: 48 - DNA, 751 bp, Artificial Sequence
  BBz platform plasmid CAR domain nucleic acid sequence
atggctctgcctgtgaccgctctgctgctgcctctggctctgctgctgcacgccgcaagacctgtcgacgcgtgctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 49 - DNA, 63 bp, Artificial Sequence
  encoding signal peptide
atggctctgcctgtgaccgctctgctgctgcctctggctctgctgctgcacgccgcaagacct

SEQ ID NO: 50 - DNA, 135 bp, Artificial Sequence
  encoding hinge region
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 51 - DNA, 72 bp, Artificial Sequence
  encoding transmembrane domain
atctacatctgggcgcccttggccgggacttgtggggtccttctcctgtcactggttatcaccctttactgc

SEQ ID NO: 52 - DNA, 126 bp, Artificial Sequence
  encoding costimulatory domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 53 - DNA, 336 bp, Artificial Sequence
  encoding intracellular signaling domain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 54 - DNA, 1458 bp, Artificial Sequence
  encoding GC33-BBz CAR structure
atggctctgcctgtgaccgctctgctgctgcctctggctctgctgctgcacgccgcaagacctgatgtcgtgatgacccagtccccactgtccctgccagtgacaccaggagagcctgcatccatctcttgccggagctcccagtctctggtgcacagcaacggcaatacctacctgcactggtatctgcagaagccaggccagagcccccagctgctgatctacaaggtgtccaaccggttctctggagtgccagaccggttcagcggctccggctctggcaccgatttcacactgaagatcagcagggtggaggcagaggacgtgggcgtgtactattgctcccagaatacccacgtgccccctacatttggccagggcaccaagctggagatcaagggaggaggaggcagcggcggaggaggctccggcggcggcggctctcaggtgcagctggtgcagtccggagcagaggtgaagaagcctggagccagcgtgaaggtgtcctgtaaggcctctggctacaccttcacagattatgagatgcactgggtgcggcaggcacctggacagggactggagtggatgggcgccctggacccaaagaccggcgatacagcctactctcagaagtttaagggcagggtgaccctgacagccgacgagagcacctccacagcctatatggagctgtctagcctgcgcagcgaggataccgccgtgtactattgcacccgcttctacagttacacttattgggggcagggcactctggtcacagtctcttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 55 - protein, 486 aa, Artificial Sequence
  GC33-BBz CAR structure
MALPVTALLLPLALLLHAARPDVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 589 aa, Artificial Sequence
  human GPC3 protein
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTETEKKIWHFKYPIFFLCIGLDLQIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSHHHHHH

SEQ ID NO: 57 - DNA, 20 bp, Artificial Sequence
  ScFv upstream operating sequence
tgctgcacgccgcaagacct

SEQ ID NO: 58 - DNA, 20 bp, Artificial Sequence
  ScFv downstream operating sequence
accacgacgccagcgccgcg

SEQ ID NO: 59 - DNA, 330 bp, Artificial Sequence
  encoding 204A VL
cagtctgtgctgacgcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatacgcgagcggcagcacactggtattcggcggagggaccaagctcaccgtccta

SEQ ID NO: 60 - DNA, 336 bp, Artificial Sequence
  encoding L1H2/L1H6 VL
gacgtggtgatgacccagtcccctctgtctttacccgttacactgggccagcccgcttccatcagctgcagaagcagccagtctttagtgcacagcaacggctacacctatttacactggtaccagcagagacccggccagagccccagactgctgatctacaaggtgtcataccgtggatccggcgtgcccgataggtttagcggcagcggtagcggcaccgatttcactttaaagatctctcgtgtcgaggccgaggatgtgggcgtttactactgcggccagagcggcctcaccccccctaccttcggctccggcaccaagctggagatcaaa

SEQ ID NO: 61 - DNA, 336 bp, Artificial Sequence
  encoding L2H6 VL
gacgtggtgatgacccagtcccctctgtctttacccgttacactgggccagcccgcttccatcagctgcagaagcagccagtctttagtgcacagcaacggctacacctatttacactggtaccagcagagacccggccagagccccagactgctgatctacaaggggtcccagcgtccctccggcgtgcccgataggtttagcggcagcggtagcggcaccgatttcactttaaagatctctcgtgtcgaggccgaggatgtgggcgtttactactgcggccagagcggcctcaccccccctaccttcggctccggcaccaagctggagatcaaa

SEQ ID NO: 62 - DNA, 360 bp, Artificial Sequence
  encoding 204A VH
gaggtgcagctggtacagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatcatacgattggggttggggcttttgatatctggggccaagggacaatggtcactgtctcttca

SEQ ID NO: 63 - DNA, 345 bp, Artificial Sequence
  encoding L1H2 VH
caagtgcagctggtgcagagcggcgccgaggtgaagaaacccggagccagcgtgaaggtgagctgtaaggccagcggctatgacttcaccgcctacgagatgcactgggtgagacaagctcccggtcaaggtctcgagtggatgggcgctttagatcccaagaccgggcagaccgcctactcccagaagttccaaggtcgtgtgactttaaccgccgacaagagcatctccaccgcctacatggagctgtctcgtctgaggagcgacgataccgtggtgtactattgcactcgtttttactcctacgcctactggggccaaggtacactggtgaccgtgagctcc

SEQ ID NO: 64 - DNA, 345 bp, Artificial Sequence
  encoding L1H6/L2H6 VH
caagtgcagctggtgcagagcggcgccgaggtgaagaaacccggagccagcgtgaaggtgagctgtaaggccagcggctatgacttcaccgcctacgagatgcactgggtgagacaagctcccggtcaaggtctcgagtggatgggcgctttagatcccaagtccggttctaccgcctactcccagaagttccaaggtcgtgtgactttaaccgccgacaagagcatctccaccgcctacatggagctgtctcgtctgaggagcgacgataccgtggtgtactattgcactcgtttctactcatacgcccactggggccaaggtacactggtgaccgtgagctcc

SEQ ID NO: 65 - DNA, 1470 bp, Artificial Sequence
  encoding 204A-BBz plasmid CAR structure
atggctctgcctgtgaccgctctgctgctgcctctggctctgctgctgcacgccgcaagacctcagtctgtgctgacgcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatacgcgagcggcagcacactggtattcggcggagggaccaagctcaccgtcctaggaggaggaggcagcggcggaggaggctccggcggcggcggctctgaggtgcagctggtacagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatcatacgattggggttggggcttttgatatctggggccaagggacaatggtcactgtctcttcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 66 - protein, 489 aa, Artificial Sequence
  204A-BBz plasmid CAR structure
MALPVTALLLPLALLLHAARPQSVLTQPASVSGSPGQSITISCTGTSSDVGGYNYVSWYQQHPGKAPKLMIYDVSNRPSGVSNRFSGSKSGNTASLTISGLQAEDEADYYCSSYASGSTLVFGGGTKLTVLGGGGSGGGGSGGGGSEVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKDHTIGVGAFDIWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 1458 bp, Artificial Sequence
  encoding L1H2-BBz plasmid CAR structure
atggctctgcctgtgaccgctctgctgctgcctctggctctgctgctgcacgccgcaagacctgacgtggtgatgacccagtcccctctgtctttacccgttacactgggccagcccgcttccatcagctgcagaagcagccagtctttagtgcacagcaacggctacacctatttacactggtaccagcagagacccggccagagccccagactgctgatctacaaggtgtcataccgtggatccggcgtgcccgataggtttagcggcagcggtagcggcaccgatttcactttaaagatctctcgtgtcgaggccgaggatgtgggcgtttactactgcggccagagcggcctcaccccccctaccttcggctccggcaccaagctggagatcaaaggaggcggaggatctggaggaggagggtctggaggcggcgggagccaagtgcagctggtgcagagcggcgccgaggtgaagaaacccggagccagcgtgaaggtgagctgtaaggccagcggctatgacttcaccgcctacgagatgcactgggtgagacaagctcccggtcaaggtctcgagtggatgggcgctttagatcccaagaccgggcagaccgcctactcccagaagttccaaggtcgtgtgactttaaccgccgacaagagcatctccaccgcctacatggagctgtctcgtctgaggagcgacgataccgtggtgtactattgcactcgtttttactcctacgcctactggggccaaggtacactggtgaccgtgagctccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 68 - protein, 486 aa, Artificial Sequence
  L1H2-BBz plasmid CAR structure
MALPVTALLLPLALLLHAARPDVVMTQSPLSLPVTLGQPASISCRSSQSLVHSNGYTYLHWYQQRPGQSPRLLIYKVSYRGSGVPDRFSGSGSGTDFTLKISRVEAEDVGVYYCGQSGLTPPTFGSGTKLEIKGGGGSGGGGSGGGGSQVQLVQSGAEVKKPGASVKVSCKASGYDFTAYEMHWVRQAPGQGLEWMGALDPKTGQTAYSQKFQGRVTLTADKSISTAYMELSRLRSDDTVVYYCTRFYSYAYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - DNA, 1458 bp, Artificial Sequence
  encoding L1H6-BBz plasmid CAR structure
atggctctgcctgtgaccgctctgctgctgcctctggctctgctgctgcacgccgcaagacctgacgtggtgatgacccagtcccctctgtctttacccgttacactgggccagcccgcttccatcagctgcagaagcagccagtctttagtgcacagcaacggctacacctatttacactggtaccagcagagacccggccagagccccagactgctgatctacaaggtgtcataccgtggatccggcgtgcccgataggtttagcggcagcggtagcggcaccgatttcactttaaagatctctcgtgtcgaggccgaggatgtgggcgtttactactgcggccagagcggcctcaccccccctaccttcggctccggcaccaagctggagatcaaaggaggcggaggatctggaggaggagggtctggaggcggcgggagccaagtgcagctggtgcagagcggcgccgaggtgaagaaacccggagccagcgtgaaggtgagctgtaaggccagcggctatgacttcaccgcctacgagatgcactgggtgagacaagctcccggtcaaggtctcgagtggatgggcgctttagatcccaagtccggttctaccgcctactcccagaagttccaaggtcgtgtgactttaaccgccgacaagagcatctccaccgcctacatggagctgtctcgtctgaggagcgacgataccgtggtgtactattgcactcgtttctactcatacgcccactggggccaaggtacactggtgaccgtgagctccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 70 - protein, 486 aa, Artificial Sequence
  L1H6-BBz plasmid CAR structure
MALPVTALLLPLALLLHAARPDVVMTQSPLSLPVTLGQPASISCRSSQSLVHSNGYTYLHWYQQRPGQSPRLLIYKVSYRGSGVPDRFSGSGSGTDFTLKISRVEAEDVGVYYCGQSGLTPPTFGSGTKLEIKGGGGSGGGGSGGGGSQVQLVQSGAEVKKPGASVKVSCKASGYDFTAYEMHWVRQAPGQGLEWMGALDPKSGSTAYSQKFQGRVTLTADKSISTAYMELSRLRSDDTVVYYCTRFYSYAHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - DNA, 1458 bp, Artificial Sequence
  encoding L2H6-BBz plasmid CAR structure
atggctctgcctgtgaccgctctgctgctgcctctggctctgctgctgcacgccgcaagacctgacgtggtgatgacccagtcccctctgtctttacccgttacactgggccagcccgcttccatcagctgcagaagcagccagtctttagtgcacagcaacggctacacctatttacactggtaccagcagagacccggccagagccccagactgctgatctacaaggggtcccagcgtccctccggcgtgcccgataggtttagcggcagcggtagcggcaccgatttcactttaaagatctctcgtgtcgaggccgaggatgtgggcgtttactactgcggccagagcggcctcaccccccctaccttcggctccggcaccaagctggagatcaaaggaggcggaggatctggaggaggagggtctggaggcggcgggagccaagtgcagctggtgcagagcggcgccgaggtgaagaaacccggagccagcgtgaaggtgagctgtaaggccagcggctatgacttcaccgcctacgagatgcactgggtgagacaagctcccggtcaaggtctcgagtggatgggcgctttagatcccaagtccggttctaccgcctactcccagaagttccaaggtcgtgtgactttaaccgccgacaagagcatctccaccgcctacatggagctgtctcgtctgaggagcgacgataccgtggtgtactattgcactcgtttctactcatacgcccactggggccaaggtacactggtgaccgtgagctccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 72 - protein, 486 aa, Artificial Sequence
  L2H6-BBz plasmid CAR structure
MALPVTALLLPLALLLHAARPDVVMTQSPLSLPVTLGQPASISCRSSQSLVHSNGYTYLHWYQQRPGQSPRLLIYKGSQRPSGVPDRFSGSGSGTDFTLKISRVEAEDVGVYYCGQSGLTPPTFGSGTKLEIKGGGGSGGGGSGGGGSQVQLVQSGAEVKKPGASVKVSCKASGYDFTAYEMHWVRQAPGQGLEWMGALDPKSGSTAYSQKFQGRVTLTADKSISTAYMELSRLRSDDTVVYYCTRFYSYAHWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR